SEQUENCE LISTING (17 sequences)

SEQ ID NO: 1 - DNA, 178 bp, homo sapiens
gggctgaatgtttcactaacaaataagaaaataaaatatttcatgtttttacagctaacaacttagtaatacctactcagagtacatacttctttatgtacccatatgaacatacaatgctatggaatgtaaagaagtatgtatttttggtaggcaataaaccaccaagggagggtac

SEQ ID NO: 2 - DNA, 174 bp, homo sapiens
ggggcaaggaggaaagatgctacaagtggcccacttctgagatgcgggctgcttctggatgacactgcttcccgaggccacatgcttctttatatccccatatggattactttgctatggaatgtaaggaagtgtgtggtttcggcaagtgcctcctcgctggccccagggtac

SEQ ID NO: 3 - DNA, 22 bp, artificial
  32P-labeled DNA oligo anti-miR-1
atacatacttctttacattcca

SEQ ID NO: 4 - DNA, 22 bp, artificial
  32P-labeled DNA oligo anti-miR-206
ccacacacttccttacattcca

SEQ ID NO: 5 - DNA, 20 bp, artificial
  32P-labeled DNA oligo anti-U6
tgtgctgccgaagcgagcac

SEQ ID NO: 6 - DNA, 22 bp, artificial
  Met-forward primer
cgctacgatgcaagagtacaca

SEQ ID NO: 7 - DNA, 21 bp, artificial
  Met-reverse primer
ttaggaaactgatcttctgga

SEQ ID NO: 8 - DNA, 21 bp, artificial
  HPRT-forward primer
tgacactggcaaaacaatgca

SEQ ID NO: 9 - DNA, 21 bp, artificial
  HPRT-reverse primer
ggtccttttcaccagcaagct

SEQ ID NO: 10 - DNA, 28 bp, artificial
  pre-miR-206 forward primer
gtccgcggggcaaggaggaaagatgcta

SEQ ID NO: 11 - DNA, 27 bp, artificial
  pre-miR-206 reverse primer
ctggtaccctggggccagcgaggaggc

SEQ ID NO: 12 - DNA, 27 bp, artificial
  pre-miR-206AS forward primer
gtccgcggctggggccagcgaggaggc

SEQ ID NO: 13 - DNA, 28 bp, artificial
  pre-miR-206AS reverse primer
ctggtaccggcaaggaggaaagatgcta

SEQ ID NO: 14 - DNA, 2346 bp, artificial
  Tpr-Met retrovirus generated subcloning the Tpr-Met cDNA
aattccgggggcggtgaggtgctgttgctgaaacgctgccgctgagggtggactcgatttcccagggtcccgccgcgggagtctccggcgggcgcgcgcgagccaccgagcgaggtgatagaggcggcggcccaggcgtctgggtcctgctggtcttcgcctttcttctccgcttctaccccgtcggccgctgccactggggtccctggccccaccgacatggcggcggtgttgcagcaagtcctggagcgcacggagctgaacaagctgcccaagtctgtccagaacaaacttgaaaagttccttgctgatcagcaatccgagatcgatggcctgaaggggcggcatgagaaatttaaggtggagagcgaacaacagtattttgaaatagaaaagaggttgtcccacagtcaggagagacttgtgaatgaaacccgagagtgtcaaagcttgcggcttgagctagagaaactcaacaatcaactgaaggcactaactgagaaaaacaaagaacttgaaattgctcaggatcgcaatattgccattcagagccaatttacaagaacaaaggaagaattagaagctgagaaaagagacttaattagaaccaatgagagactatctcaagaacttgaatacttaacagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgccatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagtagcatcagaaccagaggcttggtcccacaggccacggaccaatggcctgcagccgtgacaacactcctgtcatattggagtccaaaacttgaattccaccacactggactagtggatccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 15 - DNA, 2287 bp, homo sapiens
ggttttttttttttttttttttacaagatgttgcatcactttactttaattgcatgatttatcagaacaactattaacatacgaagtaccattcagttcagctgcaggtataggcagtgacaagtatctaattcttagaagaatcacttactcccacaatctgtccagacacattaatctaaggacaagtttataaatagcaaacgtgattttcacattgcagtgttctcaagaatgtatatacaagtgtgtagtcctgttgatgggatgtttccccgagttctttctattgatgcgttcatgctcttgaccctggtagagacagttctttctttccacagagcagattttcttttgtcatccaccatttacaatactctgtgaattagtattacttattacatttatgaaacggcaatatttggattgtaatagctcttcagtacaattccttgtgtcttctggtaagtctctgcactatcagagagcttttagttataatcattttcctgcaacaacagccaaactcaactattgagtttcagtgtgacacaccctctttgtagcttgctgggttaacccttggcttcaagtcctgatgatgtaatgagggtggggtgtattggcaatcagtacatttccttatcgcaatttacagtcattgaaaatcatgctgtcattaatcccagtctgacataccttttctaaaatgttcacagtgcagtgtttttgtggcctaacaaaatttttctcatatcattaaaaataaacatttttataaaaaatataacactttaaatgtttacgtcgacaaaaccagttagagtaacctacaccacatgcactatacagtagcaagcacaaaattccacagaatgaagcatcacaaagttctgctcagggtggctattccatctaggtgaaatagctgggattttcaattgcctttttcatttgtttctaaagtatgttttgcttaacataaaacacaccctaatgcaaaataaaactccccaaaagttttgtttccaattgcttgcgaggtgggaacctgccaccgagacagaggctaatcttttcaatccatccaccctttctttgctctacctatgagctgtgattggaaccaatgaaccttttagtaaaatgtatcctgctttacaaacatgctgagttatctttaaaaatatttatcaacaaattacttgtcttattttgagttttcatttaaaaaaatacacacaaaacatctacatgttcacattcattagatcagagtagcatcattctcaaacagtgggtttttatatgacaactaaatatttcatgatgactaaattatttcatacaaattttcttaatgtcatctatgtacattacatcctgttcatttataaacatgttttgtgtgatgctattttgaaactttgactgtattgcctataaatattcttccaaccaaaggaatgtctatacaaaaatttataagggctgggcgcagtggctcatgcctgtaatcctagcactttgggaggctgaggtgggtggatttcttgagtccaggagtttgaaaccagccttggcaacatggcaaaaccccgtctctacaaaaaatacaaaaattagccggggatggtggtgtgcgcctgtagtcccagctattcgggaggcttgagcctgggaggtggaggttgcagtgagctatgatcacaccactgcactacagccctggcaacagagtgagatcctgtctcaaaaaacaaacaaacatttaaaagggaatgtttacctaatgggtgaatgacaccatcagtaatgaaaaatgttattgaccatacaacacacaaaagtgttttgaaacactagaattctaaatgagtggcctgttctggggctgccgctcctgtcctgagcattactatctcttctgagttttctgtgatcaagaagccctcaatatttcctgcttcaatttcccatatgaaatcaagtggtacctgatttttaaaaaattcaacccagaattcaagttttggactccaatatgacaggagtgttgtcacggctgcaggccattggtccgtggcctgtgggaccaagcctctggttctgatgctctgtcagataagaaattccttagaatccagtaatttaaataacaatacaagtcctataatagtgcaattttggcaagagcaaagaatatcgatggccttttaaaggtcaggcagtgaaaaaaccattggacaaagtgtggactgttgctttgacatagtactagcaccgc

SEQ ID NO: 16 - DNA, 28 bp, artificial
  PCR forward primer for Met 3′UTR
tgccgcggatgatgaggtggacacacga

SEQ ID NO: 17 - DNA, 29 bp, artificial
  PCR reverse primer for Met 3′UTR
ctccgcggcgaagtaccattcagttcagc

(19) United States
(12) Patent Application Publication
(10) Pub. No.: US 2012/0059159 A1
(43) Pub. Date: Mar. 8, 2012

(54) DIFFERENTIATION THERAPY FOR SARCOMAS

(76) Inventors: Carola Ponzetto, Pecetto Torinese (Torino) (IT); Riccardo Taulli, Nichelino (Torino) (IT)

(21) Appl. No.: 13/320,418

(22) PCT Filed: May 25, 2009
(86) PCT No.: PCT/IB09/52179

§ 371 Date: Nov. 14, 2011

(51) Int. Cl. C07H 21/02; C12N 15/113

(52) U.S. Cl. 536/24.5

(57) ABSTRACT

Use of muscle-enriched/specific microRNAs for the treatment of sarcomas, as for example rhabdomyosarcomas, synovial sarcoma, alveolar soft part sarcoma, liposarcoma, and osteosarcoma, wherein the microRNAs are able to stop the development of neoplastic cells and to force the neoplastic cells to differentiate into terminally differentiated muscle cells.

### FIELD OF THE INVENTION

This disclosure concerns a new therapeutic approach for the treatment of sarcomas. This disclosure was devised by paying specific attention to rhabdomyosarcomas.

### TECHNICAL BACKGROUND

Rhabdomyosarcomas (RMS), the most common soft tissue sarcomas in pediatric patients and young adults, co-express markers of proliferation and myogenic differentiation. Although there is no definitive consensus on the cell of origin of RMS, it is widely believed that these tumors arise from myoblast precursors or satellite cells gone awry on their way to differentiation.

The current histological classification of RMS defines two major subtypes [embryonal (ERMS) and alveolar (ARMS)], differing in body location, occurrence, mean patient age, and prognosis. The alveolar subtype is less common but has a worse outcome, being frequently metastatic at diagnosis. While most ARMS carry the pathogenetic translocation PAX3/7-FKHR, ERMS do not carry a distinct genetic lesion and generally follow a more favorable course. The most common approach for the treatment of sarcomas is to remove—if feasible—as much tumor as possible surgically, and subsequently deliver local radiation to eradicate microscopic tumor not removed by surgery, and administer systemic (whole-body) combination chemotherapy to eradicate micrometastases. Nevertheless, many patients, especially in the case of metastatic tumors, are resistant to radiotherapy and/or chemotherapy and consequently these tumors are lethal.

### SUMMARY OF THE INVENTION

The need is therefore felt for improved solutions for the treatment of sarcomas.

The object of this disclosure is providing such improved solutions.

According to the invention, the above object is achieved thanks to the subject matter recalled specifically in the ensuing claims, which are understood as forming an integral part of this disclosure.

An embodiment of the invention provides the use of muscle-enriched/specific microRNAs for the differentiation therapy of sarcomas, such as rhabdomyosarcoma, synovial sarcoma, alveolar soft part sarcoma, liposarcoma, and osteosarcoma, wherein the microRNAs are able to stop the development of neoplastic cells by forcing them to implement the myogenic differentiation program and thus by efficiently converting them into terminally differentiated muscle tissue. The microRNAs are selected among microRNA-1, microRNA-206, or other muscle-enriched/specific microRNAs.

The present inventors have shown, as a non limiting example, that microRNA-1 and microRNA-206 have therapeutical potential in that they are able to block growth of ERMS and ARMS tumors (either at early or late stages of development) by changing the phenotype of the tumor cells from neoplastic to differentiated muscle tissue.

The present inventors have shown that miR-206 (and thus by inference miR-1 which has the same seed sequence) induces in the tumor cells a major switch in the gene expression profile toward that of mature muscle. In particular, miR-206 controls directly and indirectly the expression of close to a thousand genes, downregulating targets such as Met, which is essential for myoblast survival and replication, cell cycle genes, DNA metabolism genes and/or DNA repair genes, and up-regulating myogenic master genes such as Myo D and Myogenin as well as myofibrillar protein genes.

A further embodiment of the invention concerns the therapeutic administration of one or more muscle-enriched/specific microRNAs, among which miR-1 and miR-206 are preferred, for the treatment of a wide spectrum of tumors of mesenchymal origin, such as rhabdomyosarcomas, synovial sarcomas, alveolar soft part sarcomas, liposarcomas and osteosarcomas. Said administration would provide for stopping proliferation of sarcoma cells by down-regulating cell cycle genes and for inducing myogenic differentiation of sarcoma cells by up-regulating myogenic genes.

A further embodiment of the invention provides the use of lipid nanoparticles, liposomes or a viral vector to deliver to the sarcoma cells said microRNA(s).

### BRIEF DESCRIPTION OF THE DRAWINGS

The invention will now be described, by way of example only, with reference to the enclosed figures of drawing, wherein:

FIG. 1. MiR-1 is poorly expressed in primary tumors, and RMS cells switched to differentiating conditions fail to induce miR-1/206. (A) Quantitative real-time PCR analysis of mature miR-1 and miR-206 in primary human RMS and control muscles. Mean values (±SD) are from three replicates. (B) Increase of mature miR-1/206 in RMS cells and control human myoblasts (hMB) in differentiation medium (D, low serum) relative to proliferation medium (P, high serum), measured by real-time PCR. Mean values (±SD) are from three independent experiments. (C) Northern blot with miR-206 and miR-1-specific probes on total RNA (5 μg/lane) obtained from the indicated RMS cells grown for 3 days in differentiation medium, and from proliferating and differentiating murine satellite cells. Increasing amounts of synthetic miRNAs were used as standards for quantification. P=proliferating; D=differentiated.

FIG. 2. Conditional expression of miR-206 in RMS cells causes reduction of cell proliferation and cell cycle arrest in G0/G1, increases apoptosis, decreases invasiveness and enhances myogenic differentiation. Cells were infected with either the control (NpBI-206AS) or the miR-206-expressing (NpBI-206) vector (Tet-off) and treated (Non-Induced, NI) or not (Induced, IND.) with doxycycline (Dox). (A) proliferation of RD18 (ERMS) and RH4 (ARMS) cells was evaluated for a period of five days. The number of cells at day 0 was set at 100%. (B) cell cycle distribution of RD18 and RH4 cells in presence/absence of Dox was measured by propidium iodide staining and FACS analysis. (C) apoptosis was measured in RD18 and RH4 cells by Annexin V-APC staining and FACS analysis. Mean values (±SD) in B and C are from three independent experiments. (D) representative images of the in vitro invasion assay done on RD18 cells. (E) MHC expression in RD18 and RH4 cells upon miR-206 induction in high serum compared to controls. The level of miR-206 in NpBI-206AS IND cells was set at 1. Values represent counts of 6 fields for each group normalized against the number of DAPI-positive cells in the same fields (20×). Mean values (±SD) are from three independent experiments. Bottom: representative immunofluorescence images of induced (IND) RD18 cells carrying the NpBI-206AS and the NpBI-206 vector respectively.; bright cells=MHC. (F) Western blot of phospho-pRb, cyclin D1, phospho-p38, myogenin, p21, GFP and tubulin on Non-Induced (NI) and Induced (IND) RD18 and RH4 cells (30 μg/lane). Similar results were obtained using the Tet-on inducible system.

FIG. 3. Induction of miR-206 shifts the global gene expression profile of RMS cells towards that of muscle. (A) Unsupervised hierarchical clustering of muscles, NpBI-206 and NpBI-206AS RD18 cells (Tet-on) prior to (miR-206-non-induced; NI) and after (miR-206-induced; IND) doxycycline administration for the indicated times. Only genes showing a change>2 fold and a T-test P value<0.05 were included in the analysis. Lighter color (red in color version) indicates increased expression; darker color (blue in color version) indicates reduced expression. (B) Pearson correlation of miR-206-expressing RD18 cells (3 and 6 days) compared to normal muscle #1.

FIG. 4. MiR-206 arrests growth of RMS xenografts by promoting myogenic differentiation. (A and B) Continuous expression of pre-miR-206 (grey) prevents growth of embryonal (RD18) and alveolar (RH4) rhabdomyosarcoma xenografts. Tet-off system: half of the mice (n=5) were administered drinking water containing 1 mg/ml doxycycline starting at the time of injection (Non-Induced, NI), while the rest received water alone (Induced, IND). (C and E) Inducible expression of pre-miR-206 (grey) arrests growth of RD18 (C) and RH4 (E) xenografts. Tet-On system: 5 out of 10 mice bearing RMS tumors were given drinking water containing 1 mg/ml of doxycycline starting on the day indicated by the arrow (light grey line: pre-miR-206 induced, IND; dark grey line: antisense pre-miR-206 induced, IND). Black line: untreated controls (n=5), pre-miR-206 non-induced, NI. (D and F) miR-206 induction (Tet-on) in advanced RD18 (n=3) and RH4 (n=3) tumors is sufficient to block their growth. Tumor growth was measured every three days starting when the tumors became palpable (day 0). Bars indicate SE. (G and H) Immunohistochemical analysis of tumors harvested from doxycycline-treated animals reveals that the induction of miR-206 causes a drastic reduction of Ki67-positive cells (upper panels) and a concomitant striking increase in cells expressing the terminal differentiation marker Myosin Heavy Chain (MHC; lower panels). Pictures were taken at 20× magnification.

FIG. 5. Met is physiologically downregulated by miR-1/206 during muscle differentiation. (A) Satellite cells grown in proliferation medium (P; upper left panel) differentiate into myotubes when switched to low serum medium (D; upper right panel). Representative Northern blot of total RNA (5 μg/lane) from satellite cells (proliferating and at 3 days of differentiation) and adult murine muscles (mice # 506, 508, 582), probed for miR-1/206 expression. U6 was used as loading control. Increasing amounts of synthetic miRNAs were used as standards for quantification. (B) Western blot of extracts of satellite cells either proliferating (P) or at different stages of differentiation (day 1-4) probed for myogenin, MHC, Met and tubulin as a control. 30 μg were loaded in each lane. (C) real-time PCR on Met on the same cells. The level of Met transcript in proliferating cells was set at 100%. Mean values (±SD) are from three independent experiments.

FIG. 6. Met is directly post-transcriptionally downregulated by miR-206 targeting its 3′UTR. (A) Western blot of Met, GFP and tubulin on protein extracts (30 μg/lane) of satellite cells transfected with the Met 3′UTR reporter construct along with a scrambled or miR-206-directed LNA (400 nM) and then switched to differentiation medium for 1 to 2 days. P =proliferating; D=differentiating. (B) Western blot of Met and tubulin on protein extracts of Non-Induced (NI) and Induced (IND.) RD18 and RH4 cells (30 μg/lane).

FIG. 7. Expression of miR-206 in RMS cells impairs anchorage-independent growth and promotes terminal differentiation. Cells were infected with two different lentiviral vectors: NaldimiR-206 and NaldimiR-206 antisense (AS) as a control. (A) soft agar growth assay. The number of colonies obtained from cells infected with the control vector NaldimiR-206AS was set at 100%. Mean values (±SD) are from three independent experiments performed in triplicate. Bottom: representative images of soft agar colonies formation in cells expressing miR-206AS or miR-206. (B) graphical representation of MHC induction in RMS cells upon infection with NaldimiR-206 or with the control vector (set at 1). Values represent counts of 6 fields for each sample normalized against the number of DAPI-positive cells in the same fields (20×). Mean values (±SD) are from three independent experiments.

FIG. 8. Real-time PCR for mature miR-206 on RMS cells inducibly expressing miR-206. Cells infected with either the control (NpBI-206AS) or the miR-206-expressing (NpBI-206) vector (Tet-Off) were first sorted for high GFP expression (after a brief induction in absence of doxycycline) and then treated (Non-Induced, NI) or not (Induced, IND) with doxycycline for 3 days. Proliferating and differentiated satellite cells were included in the analysis. MiR-206 level in proliferating satellite cells was set at 1 and was used as a reference to measure the relative expression in the other samples.

FIG. 9. Tpr-Met expression decreases the ability of miR-206-expressing RD18 cells to terminally differentiate. RD18 cells stably transduced with either the control (NpBI-206AS) or the miR-206-expressing vector (NpBI-206, Tet-On) were infected with a Tpr-Met retrovirus and then treated (IND) or not (NI) with doxycicline (1 μg/ml). Myogenic differentiation was evaluated by counting MHC positive cells after 6 days of miR-206 expression. Mean values from three independent experiments (±SD) are expressed as counts of 4 fields for each group normalized by the number of DAPI-positive cells in the same fields.

### DETAILED DESCRIPTION OF EXEMPLARY EMBODIMENTS

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

The headings provided herein are for convenience only and do not interpret the scope or meaning of the embodiments.

MicroRNAs (miRNAs) are a class of highly conserved short noncoding RNAs involved in regulating cellular and developmental events. MiRNAs are initially transcribed as longer primary transcripts that undergo sequential processing by the Rnase III-like enzymes Drosha and Dicer. Mature miRNAs (21-23 nt) bind mRNAs by incomplete base pairing of their ‘seed sequence’ to complementary sequences in the 3′ untranslated region (3′UTR) of the mRNAs. Although most mRNAs targeted by miRNAs are regulated by translational repression, many of them also undergo degradation.

Numerous reports have shown that miRNAs are abnormally regulated in cancer. MiRNA genes are often located in genomic regions gained or lost in tumor cells. Some miRNAs can be functionally defined as oncogenes. However, global analysis of miRNA gene expression has revealed that miRNAs are generally downregulated in tumors compared with normal tissues. Furthermore, inhibiting miRNA processing enhances tumorigenesis, suggesting that miRNAs act mainly as oncosuppressors. Many miRNAs are expressed in a tissue-specific manner, implying important functions in differentiation. Among them, the so called myomiRs represent a well defined family, consisting of three bicistronic pairs (miR-1-1/miR-133a-2, miR-1, -2/miR-133a-1, miR-206/miR-133b). MiR-1-1 and miR-1-2 are identical and miR-206 differs from them only for three nucleotides, all outside the seed sequence. MiR-133a-2, miR-133a-1 and miR-133b are identical as well, except for one nucleotide at the 3′ end of miR-133b. Thus each of these miRNA trios can target the same mRNAs. The myomiRs are primarily involved in heart and skeletal muscle development. MiR-206, however, is the only one specific to skeletal muscle. Its expression is predominant over that of miR-1 during development and perinatally, but in adult muscle is much lower than that of miR-1.

While it has been proposed that miR-133 enhances myoblasts proliferation, there is strong evidence that miR-1 and miR-206 promote muscle differentiation. Following transfection of physiological levels of either miR-1 or miR-206, C2C12 myoblasts undergo myogenic differentiation without need for serum depletion, suggesting that these miRNAs are particularly important for induction of cell quiescence. Furthermore, forced expression of miR-1 in HeLa cells causes in the short term downregulation of hundreds of genes, most of which are expressed at low level in muscle relative to other tissues.

In the present disclosure it is showed that miR-1, which promotes myoblast differentiation, is significantly and reproducibly under-represented in primary RMS and in RMS cell lines relative to non-neoplastic muscle tissue. For its essentially identical paralog, miR-206, the present inventors were unable to obtain significant data since the variability among control samples was too high. However, it should be noted that in mature muscle the level of miR-206 is roughly 100 times lower than that of miR-1, and furthermore, that its level of expression may be affected by the relative abundance of slow versus fast-twitch fibers.

The present inventors also found that both ERMS and ARMS cell lines are unable to implement induction of these differentiative miRNAs following growth factor deprivation.

Re-adjusting miR-1/206 expression in RMS cells at a level comparable to that of differentiating satellite cells suppressed many aspects of the transformed phenotype. However, the most striking effect was the induction of myogenic differentiation, which, importantly, occurred even in the presence of growth factors. Thus, miR1-206 was sufficient to force the neoplastic cells into resuming and completing the myogenic program. Gene expression analysis via microarray revealed that miR-206 expression in RMS cells caused a major switch toward a muscle-like profile, as indicated by the fact that, among the three hundred genes found to be upregulated, many were muscle-specific, such as Myo D, Myogenin, titin, muscle creatine kinase, myosin light chain, troponin C, myomesin 1 and myosin heavy chain. Of the more than 400 downregulated genes, many were involved in the cell cycle, and DNA metabolism and repair. The time-dependency of the switch suggests that most of the observed effects of miR-206 were indirect, but among the downregulated mRNAs there were also validated [Pola1 and PTBP1] and predicted (CDK2 and CDK4) direct targets of miR-206. This finding is in line with the emerging concept that in some cases a major component of miRNA-mediated repression is mRNA destabilization.

The present inventors were particularly interested in the role of miR-1/206 on a recently validated target, the Met receptor, which is activated by overexpression in many cancers, among which RMS. They found that in normal myogenic cells at the onset of myogenesis Met is rapidly downregulated by miR-1/206 at the post-transcriptional level. Thus, failure of this molecular mechanism may underlie Met overexpression in RMS, and possibly in other types of cancer.

The inventors have previously shown that Met silencing via RNA interference reduces the oncogenicity of RMS cells in culture and in vivo, mainly by increasing apoptosis (Taulli et al, Cancer Res. 66, 4742-9). Recently, it has been described the suppressive effect of ectopic expression of miR-1 in Hepatocellular Carcinoma (HCC) and Non-Small Cell Carcinoma of the Lung (NSCLC) cells, two cancers where Met and miR-1 are also, respectively, overexpressed and underrepresented relative to the corresponding non-neoplastic tissues (Nasser et al., JBC 283,33394-485; Datta et al., Cancer Res. 68:5094-58). In these cases growth inhibition, apoptosis, and loss of tumorigenic properties were entirely ascribed to the ability of miR-1 to silence the Met receptor. Met silencing may play a major role also in the inhibition of the malignant features of RMS by miR-1/206. However, according to the present inventors, Met silencing via RNAi was more efficient than miR-1/206 in inducing apoptosis, while the latter was only mildly apoptotic but promoted myogenic differentiation. Thus, in RMS loss of the proliferative signal provided by Met leads to massive apoptosis, but when occurring in the presence of a concomitant differentiative signal it leads to differentiation.

The fact that rescue of Met signalling via its constitutively active counterpart interferes with miR-1/206-induced differentiation further confirms that Met downregulation by miR-1/206 is essential for myogenesis. Thus, sustained Met expression due to the lack of miR-1/206 contributes to the pathogenesis of RMS.

Based on the ability of miR-1/206 to act as a differentiating agent in RMS cells in culture, the present inventors proceeded to test its therapeutic potential by genetically inducing its expression in tumors derived from ERMS or ARMS cells transplanted into nude mice. Although there was no regression, the tumors stopped growing and the vast majority of their cells exited from the cell cycle and underwent terminal myogenic differentiation. The results of this experiment constitute the first in vivo proof that miR-1/206 has therapeutic potential in RMS as a differentiative agent. It should be noted that this feature overrides the remarkable genetic differences of the two RMS subtypes. Furthermore, the RMS cell lines used in the experiments have been in culture for years and carry numerous genetic lesions, among them non functional mutations of p53. Based on these results, the present inventors propose that tissue-specific miRNAs may hold greater therapeutic potential than targeted drugs, since their differentiative power is based on the ability of influencing expression of thousands of genes, and may thus not be compromised by a heterogeneous genetic landscape.

Small RNA-based therapy requires chemical modification of siRNAs and/or miRNAs to be effective in vivo. Unmodified, naked small RNAs are relatively unstable in blood and serum, as they are rapidly degraded by endo- and exonucleases, meaning that they have short half-lives in vivo. Typically, chemical modifications can be introduced into the RNA duplex structure so as to enhance biological stability without adversely affecting the gene-silencing activity. The introduction of a phosphorothioate (P=S) backbone linkage at the 3′-end protects against exonuclease degradation, and a 2′-sugar modification (such as 2′-O-methyl or 2′-fluoro) provides endonuclease resistance (Layzer et al., RNA 10, 766-71, 2004. Furthermore, siRNA and/or miRNA can be formulated with a delivery system that not only affords biological stability but also enhances cell uptake. One effective method of systemic siRNA and/or miRNA delivery involves intravenous injection of chemically modified siRNAs and/or miRNAs either conjugated to a cholesterol group or packaged into a protective liposomal particle. Cholesterol groups chemically linked to the 3′ hydroxyl group of the siRNA passenger strand facilitates cellular siRNA uptake through receptor-mediated endocytosis. Alternatively, siRNAs or microRNAs can be delivered to the target cells by means of a viral vector, which can be administered through intratumoral injection. Vectors such as Adeno-associated vectors (AAV) that do not become integrated in the host genome, are preferable compared to retroviral or lentiviral vectors, to avoid problems due to insertional mutagenesis. The limits to the use of this approach for curing cancer is that, to be effective, the anticancer microRNA has to be delivered to all of the malignant cells. Cells which escape infection will have a proliferative advantage, so the tumor may relapse, due not to resistance but to inefficient delivery of the active compound. Repeated intratumoral injections of the viral vector could be required to target the vast majority of the neoplastic cells.

Recently another group of researchers working in the field of myogenesis (Wang et al., Cancer Cell 14, 369-381, 2008) have shown that in proliferating myoblasts inhibition of “late stage myogenesis genes” is implemented, in response to high NFkB levels, by the transcriptional repressor YingYang1 (YY1), which is a target of the ubiquitously expressed miR-29. In primary RMS miR-29 is low and YY1 is increased. These researchers showed that forced expression of miR-29 in ARMS xenografts (RH30 cells) in nude mice “reduces” tumor growth and “increases” differentiation markers. They conclude suggesting that these results may have implications for the diagnosis and treatment of RMS. However, in foreseeing a possible therapeutic strategy based on miR-29 it should be kept in mind that this microRNA releases the inhibition of the expression of “late stage-myogenesis genes” while there is no evidence that it can fully re-integrate the myogenic differentiation program. Contrary to miR-29, miR-1 and miR-206 block tumor growth by acting much more ‘upstream’ in the myogenic gene cascade effectively reprogramming tumor cells into mature myogenic cells.

The present disclosure was devised by paying specific attention to rhabdomyosarcomas, but the results obtained support the therapeutic approach also for other sarcomas of mesenchymal origin, like for example synovial sarcoma, alveolar soft part sarcoma, liposarcoma and osteosarcoma.

### Materials and Methods

### Reagents

All reagents, unless specified, were from Sigma-Aldrich (St. Louis, Mo.).

miRNA

The sequences of human microRNA-1 and microRNA-206 are

miR-1 :
(SEQ ID No.: 1)
GGGCTGAATGTTTCACTAACAAATAAGAAAATAAAATATTTCATGTTT
TTACAGCTAACAACTTAGTAATACCTACTCAGAGTACATACTTCTTTAT
GTACCCATATGAACATACAATGCTATGGAATGTAAAGAAGTATGTATT
TTTGGTAGGCAATAAACCACCAAGGGAGGGTAC,
and
miR-206:
(SEQ ID No.: 2)
GGGGCAAGGAGGAAAGATGCTACAAGTGGCCCACTTCTGAGATGCGG
GCTGCTTCTGGATGACACTGCTTCCCGAGGCCACATGCTTCTTTATATC
CCCATATGGATTACTTTGCTATGGAATGTAAGGAAGTGTGTGGTTTCG
GCAAGTGCCTCCTCGCTGGCCCCAGGGTAC.

RNA sequences are presented in the form of DNA (i.e. with thymidine present instead of uracil), it is understood that these sequences are also intended to correspond to the RNA transcripts of these DNA sequences (i.e. with each T replaced by a U).

### Cell Culture, Cell Sorting and Primary Samples

RMS cells of embryonal (RD, RD18, HTB82, TE671) and alveolar (RH4, RH30) histotype, primary human myoblasts (hMB) and HEK293T were grown in DMEM (Euroclone, Pero, Italy) supplemented with 10% fetal bovine serum (FBS; Euroclone). All RMS cell lines were differentiated in DMEM with 5% horse serum (HS). hMB were differentiated in DMEM plus 4.5 mg/ml glucose, 0.5% BSA, 10 ng/ml EGF, 0.15 mg/ml creatine, 5 ng/ml insulin and 7 mM HEPES, pH 7.4. HTB82 and TE671 cells are publicly available from the Department of Experimental Medicine and Biochemichal Sciences, University of Perugia, Italy and hMB are publicly available from the Departments of Anaesthesia and Research, Basel University Hospital, Switzerland. Satellite cells were isolated from the hindlimb muscles of a 18-days old INK4a−/− mouse as previously described in Crepaldi, T., et al. (2007), J. Biol. Chem. 282: 6812-22. Proliferating cells were kept in complete growth medium [F-10 HAM containing 20% FBS, 3% chicken embryo extract (CEE) and 2.5 ng/ml basic-FGF (Peprotech, Rocky Hill, N.J.)] on gelatin-coated plates (0.5%). To obtain differentiation into myotubes, cells were plated at subconfluence on gelatin-coated plates, kept in GM for 24 hours and then switched to differentiation medium (DMEM containing 5% HS). All cells were incubated at 37° C. in a 7% CO₂-water-saturated atmosphere and media were supplemented with 2 mM L-glutamine, 100 U penicillin and 0.1 mg/ml streptomycin.

For cell sorting, cells were suspended at the concentration of 1×10⁷/ml in basic sorting buffer (5 mM EDTA, 25 mM HEPES pH 7.0, 1% heat-inactivated FBS) and then sorted for GFP expression on a MoFlo High-Performance cell sorter (DAKO Cytomation, Glostrup, Denmark).

Primary human tumors of both embryonal and alveolar histology (or their RNA) and muscle tissues were from the Memorial Sloan-Kettering Cancer Center, New York, USA and from the Division of Pediatric Pathology, Ospedale Infantile Regina Margherita, Torino, Italy following informed consent and with obscured identity for reasons of privacy. Primary murine muscles were harvested from adult mice.

### Western Blot

Cells were washed with ice-cold PBS, lysed and scraped in lysis buffer [20 mM Tris (pH 7.4), 150 mM NaCl, 5 mM EDTA, 1% Triton X-100] with 1 mM phenylmethylsulfonyl fluoride, 10 mM NaF, 1 mM Na₃VO₄, and Protease Inhibitor Cocktail. Protein lysates were cleared of cellular debris by centrifugation at 4° C. for 10 minutes at 12000×g, quantified using Bio-Rad (Hercules, Calif.) protein assay, resolved in 10% SDS-PAGE gels, and transferred to Hybond-ECL nitrocellulose membranes (Amersham Biosciences, Piscataway, N.J.). Proteins were visualized with horseradish peroxidase-conjugated secondary antibodies and Super Signal West Pico Chemiluminescent Substrate (Pierce, Rockford, Ill.).

### Antibodies

Anti Met was from Zymed (South San Francisco, Calif.); anti-cyclin D1, anti-p21 and anti-myogenin were from Santa Cruz Biotechnology (Santa Cruz, Calif.); anti-α-tubulin (B-5-1-1) was from Sigma Aldrich; anti-GFP was from Molecular Probes (Eugene, Oreg.); anti-MHC was from Developmental Studies Hybridoma Bank (University of Iowa, USA); anti-phospho-pRb and anti-phospho-p38 were from Cell Signaling Technology (Danvers, Mass.); anti-Ki67 was from Novocastra (Newcastle, United Kingdom).

### Real-time PCR and Northern Blot

RNA was extracted using Trizol (Invitrogen) for cells and snap frozen tissues and MasterPure RNA Purification Kit (Epicentre Biotechnologies) for formalin-fixed, paraffin-embedded tissues. TaqMan miRNA Assays (Applied Biosystems) were used for absolute and relative quantification of mature miR-1 and -206 expression levels. miR-16 was used to normalize the results. Reverse transcription and real-time PCR were performed according to the manufacturer's instructions. To determine absolute expression of miRNAs, a standard curve was generated using a purified RNA oligonucleotide corresponding to miR-206 (Sigma-Proligo) at the known concentrations of 10⁻³, 10⁻², 10⁻¹, 10⁰, 10¹ and 10² femtomoles. 100 nanograms of total RNA were analyzed using the Taqman miRNA Assay. Taqman cycle threshold (CT) values for each sample reaction were then converted into absolute values (femtomoles) based on the standard curve. For quantitative Northern blot analysis of miRNAs, 5 μg of total RNA were electrophoresed in a 15% polyacrylamide-urea gel and transferred by electroblotting onto Hybond-N+ membrane (Amersham). Hybridization was performed with the following 32P-labeled DNA oligos: anti-miR-1,5′-ATACATACTTCTTTACATTCCA-3′; anti-miR-206, 5′-CCACACACTTCCTTACATTCCA-3′; anti-U6, 5′-TGTGCTGCCGAAGCGAGCAC-3′. Synthetic mature miRNAs used as standards were purchased from Sigma-Proligo.

For Met detection, 1 μg of total RNA was used for reverse transcription with iScript cDNA Synthesis Kit (Bio-Rad) according to the manufacturer's protocol. Real-time PCR was performed with iQ SYBR Green (Bio-Rad) with the following primers: Met-for 5′-CGCTACGATGCAAGAGTACACA-3′, Met-rev 5′-TTAGGAAACTGATCTTCTGGA-3′, HPRT-for 5′-TGACACTGGCAAAACAATGCA-3′ and HPRT-rev 5′-GGTCCTTTTCACCAGCAAGCT-3′ as an internal control. Real-time PCR parameters were: cycle 1, 95° C. for 3 min; cycle 2, 95° C. 15 sec, 60° C. 30 sec for 40 cycles. The 2-ΔΔCT method was used to analyze the data.

### Vectors Production, Viral Transduction and LNA Transfection

NaldimiR-206 lentiviral vector was generated by PCR amplification of the pre-miR-206 locus from human genomic DNA (see above) with the following primers: pre-miR-206 for, 5′-GTCCGCGGGGCAAGGAGGAAAGATGCTA-3′ (SEQ ID No.:10) and pre-miR-206 rev, 5′-CTGGTACCCTGGGGCCAGCGAGGAGGC-3′ (SEQ ID No.:11). The PCR product was sequenced and then cloned into the SacII and KpnI sites of pCCL.sin.PPT.hPGK.GFPWpre vector (Follenzi, A., Ailles, L. E., Bakovic, S., Geuna M., and Naldini, L. (2000) Nat Genet. 25:217-22). An analogous procedure was used for NaldimiR-206AS preparation (wherein the miR206 sequence was introduced antisense, thus not functional), but with the following primers: pre-miR-206AS for 5′-GTCCGCGGCTGGGGCCAGCGAGGAGGC-3′ (SEQ ID No.:12) and pre-miR-206AS rev 5′-CTGGTACCGGCAAGGAGGAAAGATGCTA-3′ (SEQ ID No.:13). Conditional NpBI-206 and NpBI-206AS lentiviral vectors were generated by subcloning the bidirectional TRE-GFP cassette from pBI vector (Clontech, Mountain View, Calif.) into NaldimiR-206 and NaldimiR-206AS respectively between the EcoRV and SalI sites. Concentrated lentiviral vector stocks were produced as previously described in Taulli et al., (2006), Cancer Res. 66:4742-9. To obtain regulatable expression of miR-206, cells were transduced first with a lentiviral vector expressing the tTA (equivalent to pLVX-Tet-On Advanced Clontech) (for Tet-off system) or rtTA (Tet-on system) transactivator and subsequently with the responder vector NpBI-206 or NpBI-206AS. The transactivator binds to the minimal CMV promoter in absence (Tet-off) or presence (Tet-on) of doxycycline (Dox). The Tet-off inducible system enabled us to select high miR-206 expressors by sorting cells grown without Dox based on their green fluorescence. The sorted cells were then allowed to recover with Dox. Successive Dox withdrawal resulted in expression of miR-206. The Tpr-Met retrovirus was generated subcloning the Tpr-Met cDNA (SEQ ID No.:14) into blunted EcoRI and BamHI sites of Pallino retroviral vector.

Human Met 3′UTR (SEQ ID No.: 15) was PCR amplified from genomic DNA using the following primers: for 5′-TGCCGCGGATGATGAGGTGGACACACGA-3′ (SEQ ID No.:16), rev 5′-CTCCGCGGCGAAGTACCATTCAGTTCAGC-3′ (SEQ ID No.:17) and cloned downstream of GFP in the SacII restriction site of pCCL.sin.PPT.hPGK.GFPWpre lentiviral vector, that was then sequenced and used for cotransfection experiments. Pre-designed miRCURY LNA probes were purchased from Exiqon (Vedbaek, Denmark). All transfection were performed with Lipofectamine 2000 (Invitrogen) according to the manufacturer's instructions.

### Cell Proliferation Assay

Cells were seeded in 96-well plates at a density of 2×10³ cells/well. Proliferation was evaluated by MTT labeling reagent (Roche, Indianapolis, Ind.).

### Anchorage-independent Cell-Growth Assay

Cells were suspended in 0.35% type VII low melting agarose in 10% DMEM at 2×10⁴ per well and plated on a layer of 0.7% agarose in 10% DMEM in 6-well plates and cultured at 37° C. with 7% CO₂. After 2 weeks, colonies>100 μm in diameter were counted.

### Immunofluorescence

For MHC detection, cells seeded on 24-well plates and either treated or not with 1 μg/ml doxycycline for 6 days in high serum were fixed for 20 min with ice-cold methanol/acetone 2:1, washed in PBS and saturated in blocking solution (3% BSA in PBS) for 1 h. Once permeabilized with 0.3% Triton X-100 for 5 min, cells were incubated with MHC primary antibody for 1 h and then with Cy3-conjugated anti-mouse antibody (1:200) for 30 min. Nuclei were then stained with 4′,6-diamidino-2-phenylindole (DAPI). MHC and DAPI positive cells were counted with Image J at 20× magnification (6 fields/well).

### Cell Cycle Analysis

Cells were plated at a density of 1×10⁵ in 6-well plates and then treated or not with doxycycline (1 μg/ml) for 3 days. After being harvested and washed with PBS, 5×10⁵ cells were treated with RNAse (0.25 mg/ml) and stained with propidium iodide (50 μg/ml). The cell cycle distribution in G0/G1, S and G2/M phase was calculated using the CellQuest program (BD Biosciences, Franklin Lakes, N.J.).

### Assessment of Apoptosis

Apoptosis was measured by flow cytometry after staining with Annexin V. Briefly, after 5 days with or without doxycycline (1 μg/ml), cells (1×10⁵) were trypsinized, washed in PBS, and incubated for 15 minutes at 37° C. in HEPES buffer solution [10 mM HEPES (pH 7.4), 140 mM NaCl, 2.5 mM CaCl2] with 2.5 μl biotin-conjugated Annexin V (BD Biosciences). Annexin V binding was revealed by additional incubation with 0.5 μl streptavidin-allophycocyanin (APC; BD Biosciences). Cells were analyzed by FACScan using CellQuest Software (BD Biosciences).

### In Vitro Invasion Assay

Invasiveness was examined by using the membrane invasion culture system (Transwell polycarbonate membranes 6.5 mm diameter, 8 μm pore size; Corning Life Sciences, Lowell, Mass.) according to the standard protocol. Briefly, 2×10⁵ cells were seeded, in presence or absence of doxycycline (1 μg/ml), onto the upper well of transwells previously coated with 50 μl of Matrigel Basement Membrane Matrix (BD Biosciences). After 72 hrs, the noninvasive cells on the upper surface of the membrane were removed with a cotton swab. Cells migrated through the Matrigel matrix and attached to the lower surface of membrane were fixed with 11% glutaraldeyde and stained with cresyl violet.

### Microarrays and Data Analysis

Affymetrix Human GeneChip Gene ST 1.0 arrays (Affymetrix) were hybridized at the Cogentech core facility (IFOM-IEO Campus, Milano, Italy) according to standard Affymetrix protocols. 1 μg of total RNA was used as starting material for each sample. The experiment included three independent skeletal muscles (n=3), and six biological replicates of RD18 cells previously infected with the inducible NpBI-206 vector and then treated or not with doxycycline (Tet-on), thus giving rise to both miR-206-induced (n=4) and miR-206-non-induced (n=2) cells. Moreover, NpBI-206AS infected cells were used as additional controls in both induced and non-induced conditions. The array data were analyzed with the Partek Genomics Suite v6.3 software (Partek Inc.). All 734 genes showing differential expression between the two exprimental conditions in RD18 cells and found to be significant by ANOVA (fold change compared to the mean across the whole panel>2 and T-test P value<0.05) were then subjected to unsupervised hierarchical clustering. Normal muscle samples were also included in the clustering. The same set of up- and downregulated genes was further analyzed to reveal enrichment of functional categories using the Database for Annotation, Visualization and Integrated Discovery (DAVID) 2008 (http://david.abcc.ncifcrf.gov/). We used the Functional Annotation Tool program and reported only GOTERM-BP (Biological Process) that had P values<0.05 after correcting for multiple testing (Benjamini-Hochberg). The EIMMo miRNA target prediction server (http://www.mirz.unibas.ch/E1MMo2/) was used to identify putative miR-206 targets among the downregulated transcripts in miR-206-induced compared to non-induced RD18 cells.

### In Vivo Tumorigenesis Assay

Cells were trypsinized and resuspended at 1×10⁷ cells/ml in sterile PBS. 200 μl were injected subcutaneously into the flank of female nu/nu mice (Charles River, Wilmington, Mass.). Tumor size was measured with Vernier calibers every 3 days, and tumor volumes were calculated as the volume of a sphere. Conditional miR-206 expression was induced in mice with (Tet-on system) or without (Tet-off system) 1 mg/ml doxycycline in the drinking water. It has to be noted that with the Tet-off system it was observed a 2-weeks lag time after induction before GFP expression. Besides, fluorescence was rather weak and spotty in the tumors. On the contrary, GFP was detectable after just 48 hours of induction with the Tet-on system, that was therefore chosen for the experiments where miR-206 was induced in already palpable tumors.

### Immunohistochemistry

Tumor samples were collected at the indicated times, fixed for 2 hours in 4% paraformaldehyde and embedded in paraffin. Rehydrated sections were treated with 3% H₂O₂ and microwaved for 30 min in 10 mM Antigen Retrieval Citra (Biogenex, San Ramon, Calif.). All antibodies incubation were performed at room temperature with the solutions provided by the DakoCytomation LSAB2 System-HRP kit (Dako, Carpinteria, Calif.; primary antibody: 1 h; peroxidase-conjugated secondary antibody: 30 min). Staining was developed by liquid diaminobenzidine chromogen (Biogenex) followed by hematoxylin.

### Results

MiR-1 Expression is Low in Primary RMS, and RMS Cells Fail to Induce miR-1 and miR-206 Upon Being Switched to Differentiation Medium

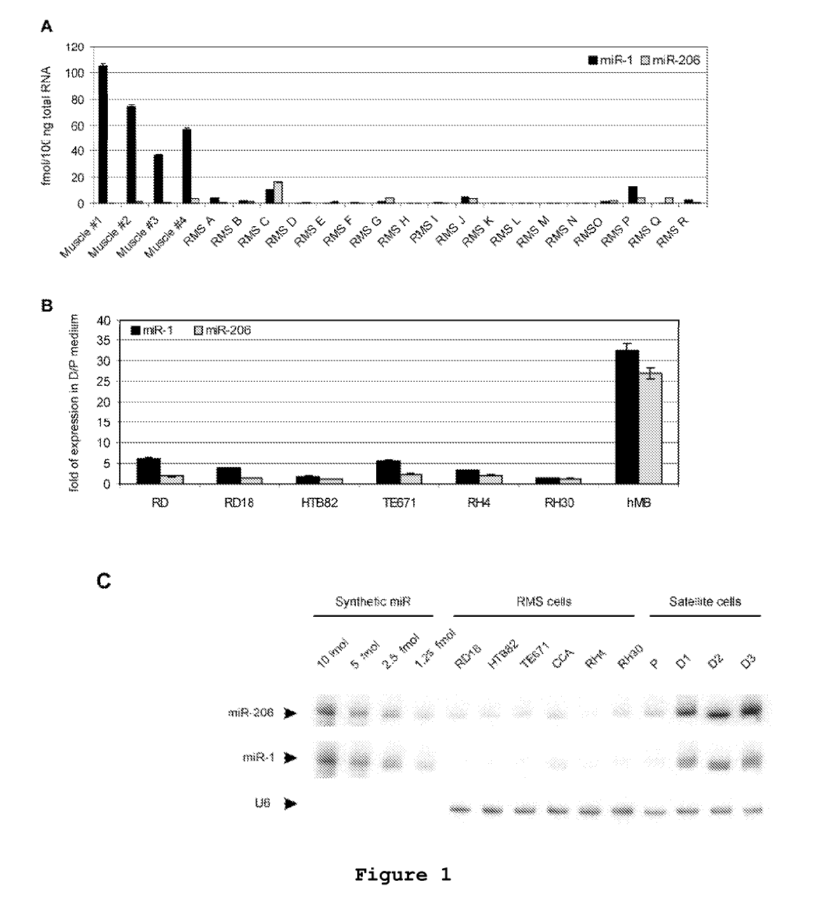

Recently, in a survey of miRNA expression signatures in human sarcomas, miR-1 was found to be under-represented relative to normal muscle in 3 ARMS and 2 PRMS (pleiomorphic RMS). The level of expression of miR-1 and miR-206 was determined in four control muscles and in a panel of primary RMS, including 10 ERMS e 8 ARMS. As expected, in control muscle the absolute level of miR-1 was, on the average, over sixty times that of miR-206. In line with previous reports expression of miR-1 in RMS tumors was absent or of the same order as miR-206 (FIG. 1A). Considering that miR-1 and miR-206 are normally strongly upregulated at the onset of myogenesis, it was verified whether their level of expression in ERMS and ARMS cell lines changed upon switching from proliferating to differentiating conditions. It has been found that in RMS cells induction of these myomiRs was reduced or absent with respect to primary human myoblasts (FIG. 1B). This result was confirmed by Northern blot. In all RMS cells switched to differentiation medium the levels of miR-1 and miR-206 remained very low, of the order of those found in proliferating myoblasts (FIG. 1C).

Rescue of miR-1/206 Expression in ERMS and ARMS Cell Lines Interferes with the Transformed Phenotype by Promoting Terminal Differentiation

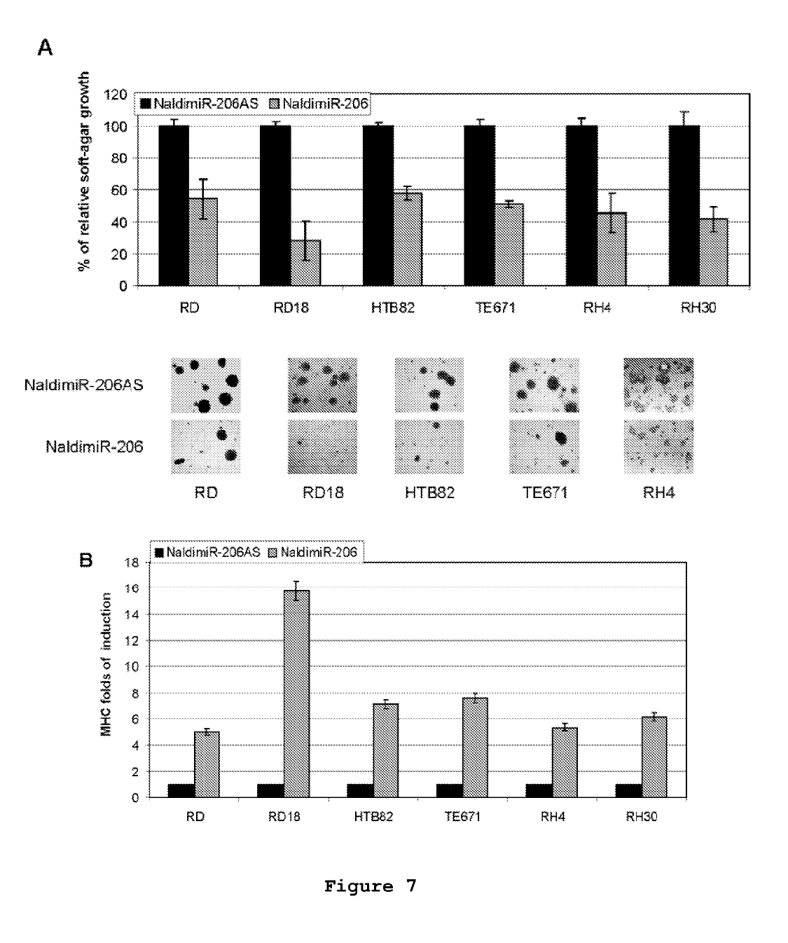

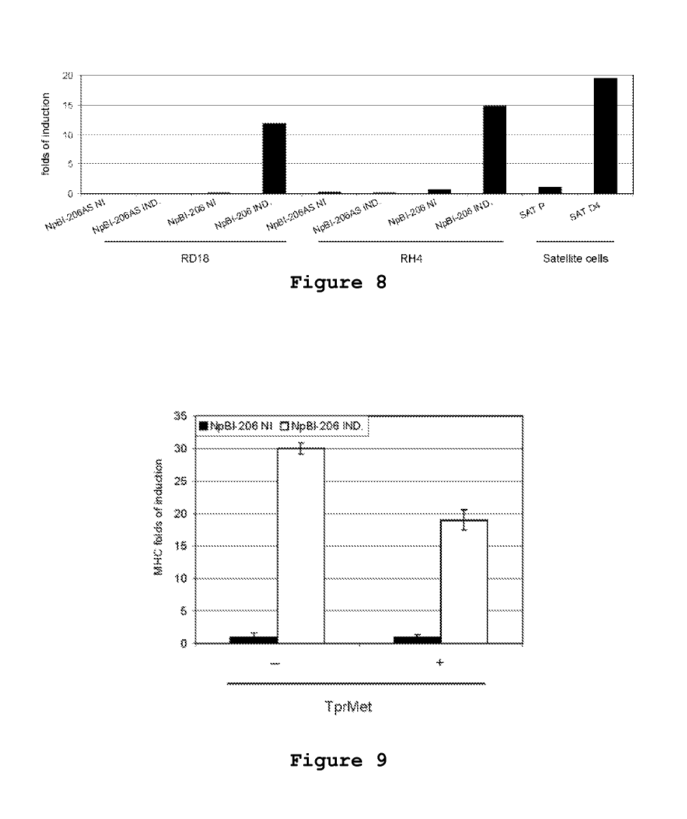

To verify whether the inability to upregulation miR-1 and -206 was responsible for blocked differentiation typical of rhabdomyosarcoma, miR-1 and -206 were reintroduced in RMS cells. To this end lentiviral vectors constitutively expressing pre-miR-1 and -206 along with GFP were produced. Since the two miRNAs are virtually identical and miR-206 was expressed more efficiently than miR-1, it has been chosen to continue the present studies using miR-206, hereafter defined miR-1/206. Rescue of miR-1/206 expression (evidenced by the green fluorescence of the reporter) caused in all RMS cell lines a ˜50% reduction in soft agar colony formation (FIG. 7A). Furthermore, quantification of the cells positive for the differentiated muscle marker myosin heavy chain (MHC), showed that their number, very low in the cultures infected with the control vector, increased significantly a few days after miR-1/206 transduction (FIG. 7B). However, keeping the cells in culture for a longer time, resulted in positive selection of those with low or no GFP, which ultimately out-numbered those expressing GFP/miR-1/206. Thus, especially in view of testing in vivo the effects of miR-1/206, it was set up an inducible system. This allowed to select miR-1/206 expressors by sorting them, following a brief pulse of induction, on the basis of their green fluorescence (FIG. 8).

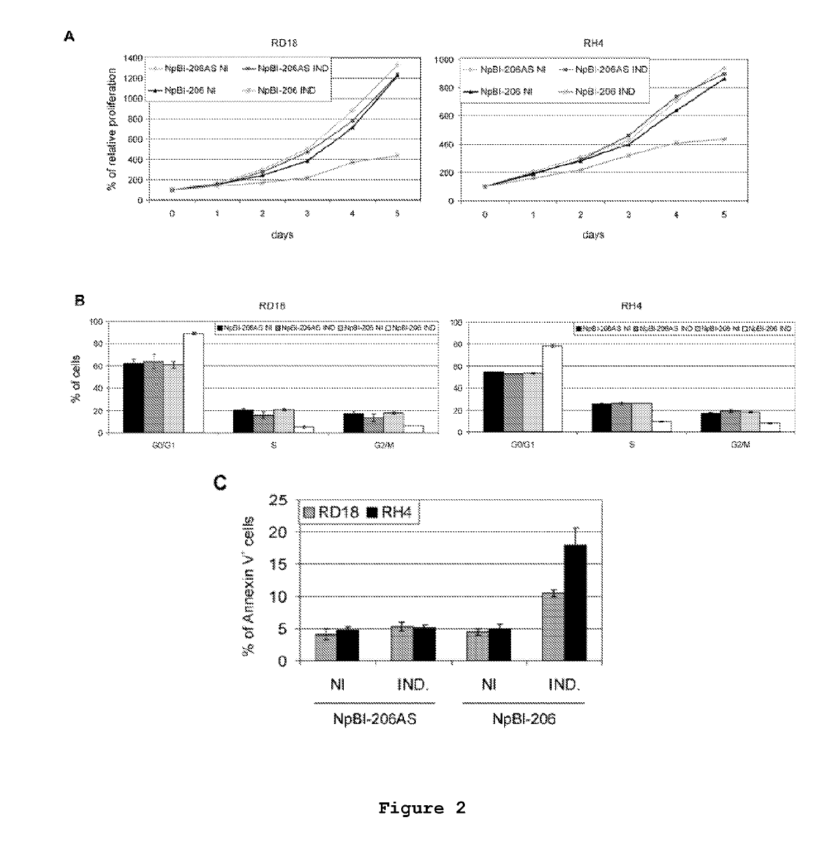

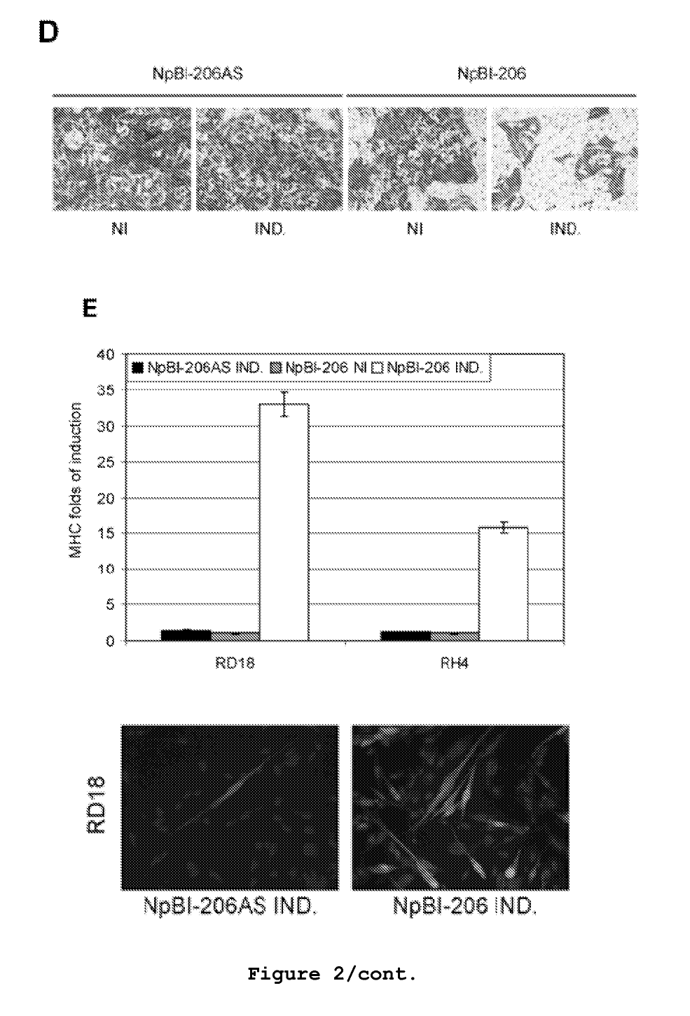

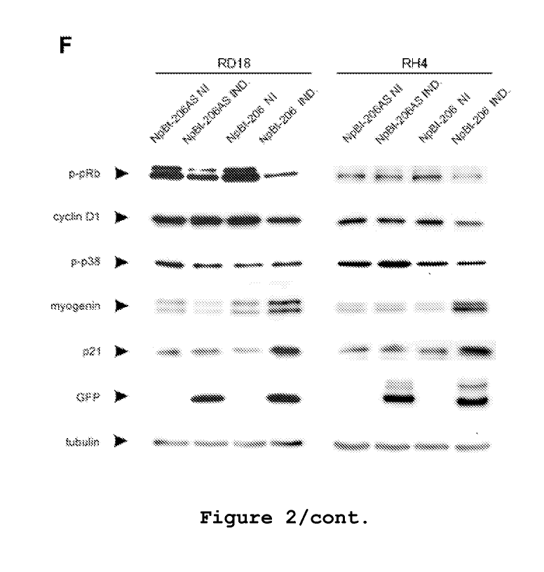

Recovery of miR-1/206 expression using the inducible system at a level approaching that of differentiating satellite cells (FIG. 8) caused a strong reduction of the proliferation rate in both ERMS and ARMS cells (FIG. 2A). Accordingly, cell cycle analysis done 72 hours after miR-206 induction showed an accumulation in G0/G1 and a concomitant reduction in phase S and G2/M (FIG. 2B). miR-1/206 also promoted apoptosis, as shown by Annexin V staining (FIG. 2C), and decreased Matrigel invasiveness (FIG. 2D). Six days after miR-1/206 induction, the number of MHC-positive cells in ERMS and ARMS cultures increased about 30 and 15 fold, respectively (FIG. 2E upper panel). MHC-positive cells were often elongated and multinucleate (FIG. 2E lower panels), indicating terminal myogenic differentiation. To explore the effect of rescuing miR-1/206 on key molecules of the myogenic lineage, it was used Western blot to analyze gene products involved in cell proliferation and differentiation. While phosphorylation of p38 MAPK was not affected, miR-1/206 expression caused downregulation of cyclin D1 and phospho-pRb and upregulation of p21 and myogenin (FIG. 2F). These changes were consistent with exit from the cell cycle and activation of the myogenic program.

Induction of miR-1/206 Expression Promotes RMS Differentiation by Modulating More than 700 Genes

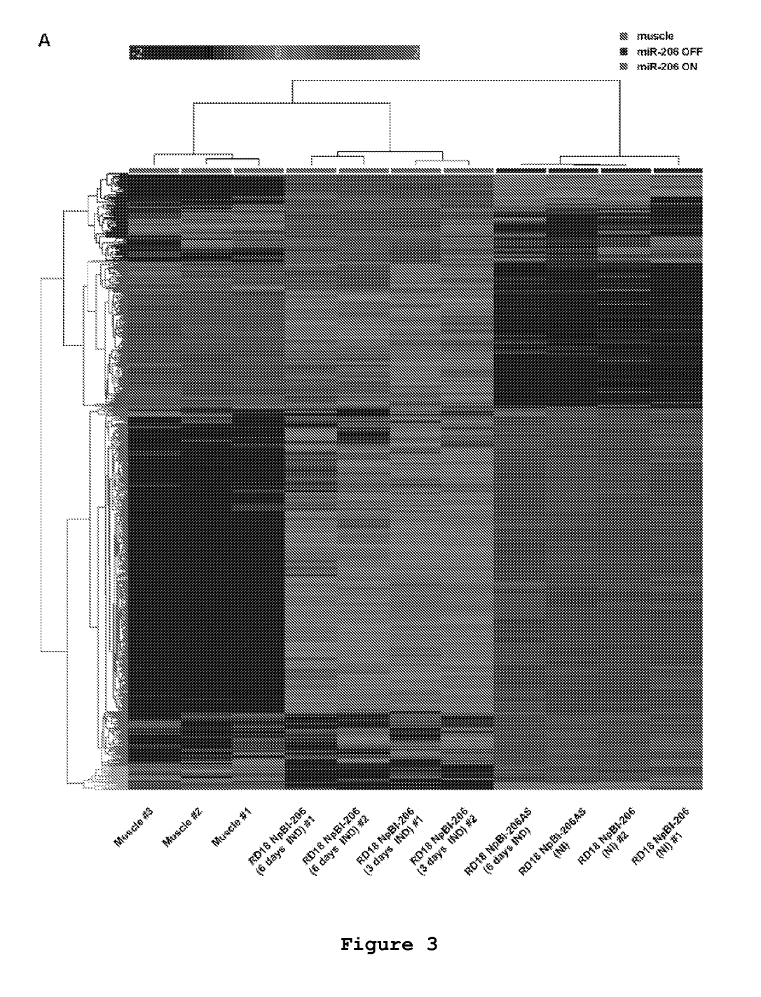

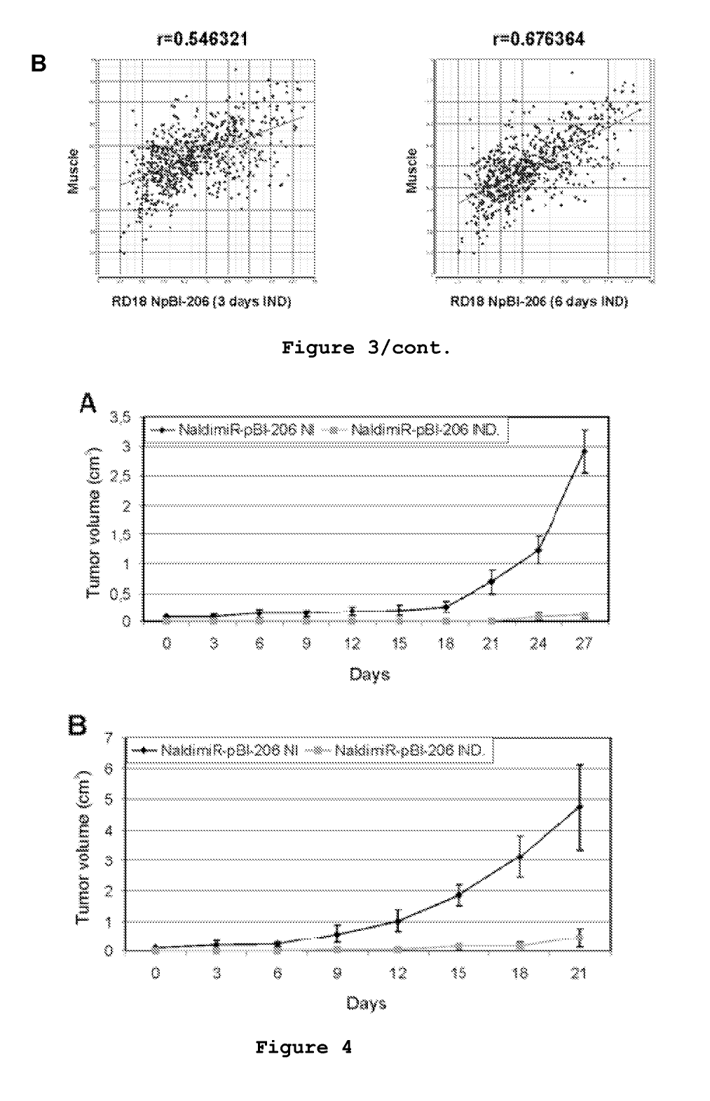

To substantiate the above conclusion at the level of global gene expression, the long-term changes in the mRNA profile of RD18 cells was determined before (NI) and after (IND) miR-206 induction using microarrays (FIG. 3A). As a control RD18 cells transduced with the inducible vector expressing miR-206 in antisense were used. It was chosen to focuse on the 734 genes more significantly induced (278, violet cluster in the vertical axis of the dendrogram) or repressed (456, green cluster in the vertical axis of the dendrogram) after doxycycline treatment. Unsupervised hierarchical clustering (including also the data from three normal skeletal muscles biopsies) generated a dendrogram with 2 major branches, one of which contained the NI miR-206 and both the NI and IND miR-206AS RD18 cells, while the second one grouped both normal muscles and RD18 cells where miR-206 expression was induced for 3 and 6 days, respectively. The results of this experiment indicated that, on the whole, expression of miR-206 indeed shifted the global gene expression profile of RMS cells towards that of differentiated muscle, with the exception of two minor clusters of genes (blue and yellow in the vertical axis of the dendrogram), which after induction were differentially expressed with respect to mature muscle.

The extent of the RMS-to-muscle shift in gene expression depended on the length of miR-206 induction. In fact the Pearson correlation between RD18 cells and muscle increased from 0.01 in NI cells (not shown) to 0.55 and 0.68 in cells treated with doxycycline for, respectively, 3 and 6 days (FIG. 3B). To characterize the genes that were modulated by miR-206 in RD18 cells, we assigned them to functional categories. The more significantly upregulated genes (P value<0.05) were enriched for muscle-related functions, while the more significantly downregulated included genes involved in the control of cell cycle, metabolism, and DNA repair (Table 1 and Table 2). Using the EIMMo miRNA target prediction server (http://www.mirz.unibas.ch/E1MMo2/) we found among the downregulated genes a number of predicted miR-206 targets, including Met. A list is provided in Table 3. In sum, the microarray analysis provided strong evidence for the induction of muscle differentiation upon expression of miR-206 in RMS cells.

TABLE 1
Top 12 enriched functional categories of genes modulated by
miR-206 induction in RD18 cells (corrected P value < 0.05).

| Category | P value |
|---|---|
| Upregulated genes | |
| muscle system process | 5.80E−13 |
| muscle contraction | 5.80E−13 |
| muscle development | 1.00E−05 |
| regulation of muscle contraction | 3.00E−03 |
| system process | 2.40E−02 |
| system development | 2.80E−02 |
| striated muscle contraction | 3.10E−02 |
| anatomical structure development | 3.10E−02 |
| cellular component organization and biogenesis | 4.30E−02 |
| organ development | 3.90E−02 |
| multicellular organismal process | 3.60E−02 |
| Downregulated genes | |
| cell cycle | 1.30E−65 |
| cell cycle process | 7.10E−58 |
| M phase | 8.80E−58 |
| mitosis | 2.40E−57 |
| M phase of mitotic cell cycle | 3.70E−57 |
| cell cycle phase | 3.60E−57 |
| mitotic cell cycle | 5.40E−56 |
| cell division | 8.10E−54 |
| DNA metabolic process | 1.20E−45 |
| DNA replication | 8.50E−32 |
| organelle organization and biogenesis | 2.40E−24 |

TABLE 2
Comprehensive list of the enriched functional categories of genes
modulated by miR-206 induction in RD18 cells with a corrected P value < 0.05.

| | | Corrected |
|---|---|---|
| Category | Term | P value |
| UPREGULATED GENES | | |
| GOTERM_BP_ALL | muscle system process | 5.80E−13 |
| GOTERM_BP_ALL | muscle contraction | 5.80E−13 |
| GOTERM_BP_ALL | muscle development | 1.00E−05 |
| GOTERM_BP_ALL | regulation of muscle contraction | 3.00E−03 |
| GOTERM_BP_ALL | system process | 2.40E−02 |
| GOTERM_BP_ALL | system development | 2.80E−02 |
| GOTERM_BP_ALL | striated muscle contraction | 3.10E−02 |
| GOTERM_BP_ALL | anatomical structure development | 3.10E−02 |
| GOTERM_BP_ALL | cellular component organization and biogenesis | 4.30E−02 |
| GOTERM_BP_ALL | organ development | 3.90E−02 |
| GOTERM_BP_ALL | multicellular organismal process | 3.60E−02 |
| DOWNREGULATED GENES | | |
| GOTERM_BP_ALL | cell cycle | 1.30E−65 |
| GOTERM_BP_ALL | cell cycle process | 7.10E−58 |
| GOTERM_BP_ALL | M phase | 8.80E−58 |
| GOTERM_BP_ALL | mitosis | 2.40E−57 |
| GOTERM_BP_ALL | M phase of mitotic cell cycle | 3.70E−57 |
| GOTERM_BP_ALL | cell cycle phase | 3.60E−57 |
| GOTERM_BP_ALL | mitotic cell cycle | 5.40E−56 |
| GOTERM_BP_ALL | cell division | 8.10E−54 |
| GOTERM_BP_ALL | DNA metabolic process | 1.20E−45 |
| GOTERM_BP_ALL | DNA replication | 8.50E−32 |
| GOTERM_BP_ALL | organelle organization and biogenesis | 2.40E−24 |
| GOTERM_BP_ALL | regulation of progression through cell cycle | 8.30E−24 |
| GOTERM_BP_ALL | regulation of cell cycle | 1.00E−23 |
| GOTERM_BP_ALL | response to DNA damage stimulus | 1.50E−23 |
| GOTERM_BP_ALL | chromosome organization and biogenesis | 8.50E−22 |
| GOTERM_BP_ALL | DNA repair | 5.20E−21 |
| GOTERM_BP_ALL | response to endogenous stimulus | 1.20E−19 |
| GOTERM_BP_ALL | chromosome segregation | 1.40E−18 |
| GOTERM_BP_ALL | DNA-dependent DNA replication | 1.30E−16 |
| GOTERM_BP_ALL | cell cycle checkpoint | 6.20E−16 |
| GOTERM_BP_ALL | nucleobase, nucleoside, nucleotide and nucleic acid | 5.80E−15 |
| | metabolic process | |
| GOTERM_BP_ALL | regulation of mitosis | 7.20E−15 |
| GOTERM_BP_ALL | nucleosome assembly | 5.10E−14 |
| GOTERM_BP_ALL | chromatin assembly | 7.30E−14 |
| GOTERM_BP_ALL | biopolymer metabolic process | 1.90E−13 |
| GOTERM_BP_ALL | mitotic sister chromatid segregation | 1.00E−12 |
| GOTERM_BP_ALL | sister chromatid segregation | 1.70E−12 |
| GOTERM_BP_ALL | microtubule-based process | 2.40E−12 |
| GOTERM_BP_ALL | spindle organization and biogenesis | 2.60E−12 |
| GOTERM_BP_ALL | chromatin assembly or disassembly | 8.50E−12 |
| GOTERM_BP_ALL | cellular component organization and biogenesis | 3.00E−11 |
| GOTERM_BP_ALL | DNA packaging | 1.00E−10 |
| GOTERM_BP_ALL | protein-DNA complex assembly | 2.50E−10 |
| GOTERM_BP_ALL | cellular process | 3.80E−10 |
| GOTERM_BP_ALL | interphase | 6.30E−10 |
| GOTERM_BP_ALL | DNA replication initiation | 8.90E−10 |
| GOTERM_BP_ALL | establishment and/or maintenance | 1.80E−09 |
| | of chromatin architecture | |
| GOTERM_BP_ALL | interphase of mitotic cell cycle | 2.40E−09 |
| GOTERM_BP_ALL | mitotic cell cycle checkpoint | 4.60E−08 |
| GOTERM_BP_ALL | cytoskeleton organization and biogenesis | 6.30E−08 |
| GOTERM_BP_ALL | microtubule cytoskeleton organization and biogenesis | 6.50E−08 |
| GOTERM_BP_ALL | macromolecular complex assembly | 3.00E−07 |
| GOTERM_BP_ALL | macromolecule metabolic process | 4.30E−07 |
| GOTERM_BP_ALL | cellular component assembly | 5.10E−07 |
| GOTERM_BP_ALL | primary metabolic process | 2.90E−06 |
| GOTERM_BP_ALL | response to stress | 3.60E−06 |
| GOTERM_BP_ALL | cellular metabolic process | 4.20E−06 |
| GOTERM_BP_ALL | cell proliferation | 6.30E−06 |
| GOTERM_BP_ALL | chromosome condensation | 8.50E−06 |
| GOTERM_BP_ALL | phosphoinositide-mediated signaling | 1.20E−05 |
| GOTERM_BP_ALL | regulation of progression through mitotic cell cycle | 1.20E−05 |
| GOTERM_BP_ALL | microtubule-based movement | 2.20E−05 |
| GOTERM_BP_ALL | mitotic chromosome condensation | 3.10E−05 |
| GOTERM_BP_ALL | regulation of cyclin-dependent protein kinase activity | 4.30E−05 |
| GOTERM_BP_ALL | cytoskeleton-dependent intracellular transport | 1.30E−04 |
| GOTERM_BP_ALL | establishment of chromosome localization | 1.70E−04 |
| GOTERM_BP_ALL | chromosome localization | 1.70E−04 |
| GOTERM_BP_ALL | DNA integrity checkpoint | 1.70E−04 |
| GOTERM_BP_ALL | traversing start control point of mitotic cell cycle | 3.80E−04 |
| GOTERM_BP_ALL | centrosome duplication | 3.80E−04 |
| GOTERM_BP_ALL | centrosome cycle | 5.40E−04 |
| GOTERM_BP_ALL | regulation of DNA metabolic process | 5.70E−04 |
| GOTERM_BP_ALL | spindle checkpoint | 7.10E−04 |
| GOTERM_BP_ALL | metabolic process | 1.80E−03 |
| GOTERM_BP_ALL | microtubule organizing center organization | 1.80E−03 |
| | and biogenesis | |
| GOTERM_BP_ALL | centrosome organization and biogenesis | 1.80E−03 |
| GOTERM_BP_ALL | cytokinesis | 2.60E−03 |
| GOTERM_BP_ALL | meiosis | 2.70E−03 |
| GOTERM_BP_ALL | M phase of meiotic cell cycle | 2.70E−03 |
| GOTERM_BP_ALL | meiotic cell cycle | 3.30E−03 |
| GOTERM_BP_ALL | double-strand break repair | 3.40E−03 |
| GOTERM_BP_ALL | mitotic spindle organization and biogenesis | 4.10E−03 |
| GOTERM_BP_ALL | establishment of organelle localization | 1.00E−02 |
| GOTERM_BP_ALL | DNA unwinding during replication | 1.10E−02 |
| GOTERM_BP_ALL | DNA damage checkpoint | 1.40E−02 |
| GOTERM_BP_ALL | DNA synthesis during DNA repair | 1.60E−02 |
| GOTERM_BP_ALL | mitotic cell cycle spindle assembly checkpoint | 1.60E−02 |
| GOTERM_BP_ALL | DNA geometric change | 1.70E−02 |
| GOTERM_BP_ALL | DNA duplex unwinding | 1.70E−02 |
| GOTERM_BP_ALL | regulation of DNA replication | 1.80E−02 |
| GOTERM_BP_ALL | DNA damage response, signal transduction | 1.80E−02 |
| GOTERM_BP_ALL | second-messenger-mediated signaling | 1.90E−02 |
| GOTERM_BP_ALL | S phase | 3.00E−02 |
| GOTERM_BP_ALL | G1 phase of mitotic cell cycle | 3.00E−02 |
| GOTERM_BP_ALL | negative regulation of DNA metabolic process | 3.00E−02 |
| GOTERM_BP_ALL | organelle localization | 3.40E−02 |
| GOTERM_BP_ALL | regulation of kinase activity | 3.70E−02 |
| GOTERM_BP_ALL | regulation of transferase activity | 4.40E−02 |

TABLE 3
Comprehensive list of the predicted miR-206 targets found among the
downregulated transcripts in miR-206-induced compared to non-induced RD18 cells.

| RefSeq | | EIMMo | Location of the seed matches |
|---|---|---|---|
| transcript ID | Gene description | score (*) | within the 3′UTR |
| NM_014445 | Homo sapiens stress-associated endoplasmic reticulum protein 1 | 2.0213 | 3′UTR -X------X---- |
| | (SERP1), mRNA. | | |
| NM_016395 | Homo sapiens protein tyrosine phosphatase-like A domain | 1.8954 | 3′UTR -X------X-- |
| | containing 1 (PTPLAD1), mRNA | | |
| NM_032826 | Homo sapiens solute carrier family 35, member B4 (SLC35B4), | 1.8726 | 3′UTR ---------X----------X- |
| | mRNA. | | XX--X |
| NM_000165 | Homo sapiens gap junction protein, alpha 1, 43 kDa (GJA1), 1 | 1.7662 | 3′UTR --X-----X- |
| | mRNA. | | |
| NM_000402 | Homo sapiens glucose-6-phosphate dehydrogenase (G6PD), | 1.7147 | 3′UTR XXX- |
| | transcript variant 1, mRNA. | | |
| NM_017542 | Homo sapiens pogo transposable element with KRAB domain 1 | 1.7019 | 3′UTR -----XXX--- |
| | (POGK), mRNA. | | |
| NM_015171 | Homo sapiens exportin 6 (XPO6), mRNA. | 1.4451 | 3′UTR --X- |
| NM_000245 | Homo sapiens met proto-oncogene (hepatocyte growth factor | 1.3449 | 3′UTR --X-X------- |
| | receptor) (MET), transcript variant 2, mRNA. | | |
| NM_000408 | Homo sapiens glycerol-3-phosphate dehydrogenase 2 | 1.1701 | 3′UTR -X----------X------ |
| | (mitochondrial) (GPD2), nuclear gene encoding mitochondrial | | |
| | protein, transcript variant 2, mRNA. | | |
| NM_031991 | Homo sapiens polypyrimidine tract binding protein 1 (PTBP1), | 1.1417 | 3′UTR X------X- |
| | transcript variant 3, mRNA. | | |
| NM_002819 | Homo sapiens polypyrimidine tract binding protein 1 (PTBP1), | 1.1417 | 3′UTR X------X- |
| | transcript variant 1, mRNA. | | |
| NM_031990 | Homo sapiens polypyrimidine tract binding protein 1 (PTBP1), | 1.1417 | 3′UTR X------X- |
| | transcript variant 2, mRNA. | | |
| NM_175847 | Homo sapiens polypyrimidine tract binding protein 1 (PTBP1), | 1.1417 | 3′UTR X------X- |
| | transcript variant 4, mRNA. | | |
| XM_942692 | PREDICTED: Homo sapiens hypothetical protein MGC5139, | 1.1341 | 3′UTR ------------X----XX |
| | transcript variant 3 (MGC5139), mRNA. | | |
| NM_006148 | Homo sapiens LIM and SH3 protein 1 (LASP1), mRNA. | 1.1037 | 3′UTR X-X------------- |
| NM_002823 | Homo sapiens prothymosin, alpha (gene sequence 28) (PTMA), | 0.8649 | 3′UTR --X-- |
| | transcript variant 2, mRNA. | | |
| NM_016271 | Homo sapiens ring finger protein 138 (RNF138), transcript 1 | 0.8436 | 3′UTR -------X----- |
| | variant 1, mRNA. | | |
| NM_198128 | Homo sapiens ring finger protein 138 (RNF138), transcript | 0.8436 | 3′UTR -------X----- |
| | variant 2, mRNA. | | |
| NM_003769 | Homo sapiens splicing factor, arginine/serine-rich 9 (SFRS9), | 0.8436 | 3′UTR X-- |
| | mRNA. | | |
| NM_015840 | Homo sapiens adenosine deaminase, RNA-specific (ADAR), | 0.8206 | 3′UTR --------------X |
| | transcript variant 2, mRNA. | | |
| NM_015841 | Homo sapiens adenosine deaminase, RNA-specific (ADAR), | 0.8206 | 3′UTR --------------X |
| | transcript variant 3, mRNA. | | |
| NM_001025107 | Homo sapiens adenosine deaminase, RNA-specific (ADAR), | 0.8206 | 3′UTR --------------X |
| | transcript variant 4, mRNA. | | |
| NM_001111 | Homo sapiens adenosine deaminase, RNA-specific (ADAR), | 0.8206 | 3′UTR --------------X |
| | transcript variant 1, mRNA. | | |
| NM_201279 | Homo sapiens neuropilin 2 (NRP2), transcript variant 3, mRNA. | 0.8206 | 3′UTR X--------------- |
| NM_003872 | Homo sapiens neuropilin 2 (NRP2), transcript variant 2, mRNA. | 0.8206 | 3′UTR X--------------- |
| NM_201266 | Homo sapiens neuropilin 2 (NRP2), transcript variant 1, mRNA. | 0.8206 | 3′UTR X--------------- |
| NM_018290 | Homo sapiens phosphoglucomutase 2 (PGM2), mRNA. | 0.8204 | 3′UTR -X------ |
| NM_006367 | Homo sapiens CAP, adenylate cyclase-associated protein 1 | 0.8151 | 3′UTR ---X--- |
| | (yeast) (CAP1), transcript variant 1, mRNA. | | |
| NM_030920 | Homo sapiens acidic (leucine-rich) nuclear phosphoprotein 32 | 0.7940 | 3′UTR -X---------- |
| | family, member E (ANP32E), transcript variant 1, mRNA. | | |
| NM_006826 | Homo sapiens tyrosine 3-monooxygenase/tryptophan 5- | 0.7685 | 3′UTR --X----- |
| | monooxygenase activation protein, theta polypeptide | | |
| | (YWHAQ), mRNA. | | |
| NM_004965 | Homo sapiens high-mobility group nucleosome binding | 0.7484 | 3′UTR -X--- |
| | domain 1 (HMGN1), mRNA. | | |
| NM_018092 | Homo sapiens neuropilin (NRP)and tolloid (TLL)-like 2 | 0.7270 | 3′UTR --X-X---- |
| | (NETO2), mRNA. | | |
| NM_138340 | Homo sapiens abhydrolase domain containing 3 (ABHD3), | 0.7195 | 3′UTR --X-- |
| | mRNA. | | |
| NM_001064 | Homo sapiens transketolase (Wernicke-Korsakoff syndrome) | 0.6725 | 3′UTR X- |
| | (TKT), mRNA. | | |
| NM_006401 | Homo sapiens acidic (leucine-rich) nuclear phosphoprotein 32 | 0.6725 | 3′UTR -X-- |
| | family, member B (ANP32B), mRNA. | | |
| NM_152259 | Homo sapiens chromosome 15 open reading frame 42 | 0.6724 | 3′UTR -X---- |
| | (C15orf42), mRNA. | | |
| NM_000274 | Homo sapiens ornithine aminotransferase (gyrate atrophy) | 0.6591 | 3′UTR X--X |
| | (OAT), nuclear gene encoding mitochondrial protein, mRNA. | | |
| NM_006145 | Homo sapiens DnaJ (Hsp40) homolog, subfamily B, member 1 | 0.6384 | 3′UTR -X----- |
| | (DNAJB1), mRNA. | | |
| NM_018375 | Homo sapiens solute carrier family 39 (zinc transporter), | 0.6093 | 3′UTR ---XX------ |
| | member 9 (SLC39A9), mRNA. | | |
| NM_016937 | Homo sapiens polymerase (DNA directed), alpha 1 (POLA1), | 0.5803 | 3′UTR ---X-- |
| | mRNA. | | |
| NM_005497 | Homo sapiens gap junction protein, alpha 7, 45 kDa (GJA7), | 0.5569 | 3′UTR -----X---X---------------- |
| | transcript variant 1, mRNA. | | ------ |
| NM_001008744 | Homo sapiens tyrosyl-DNA phosphodiesterase 1 (TDP1), | 0.5569 | 3′UTR -X--X---- |
| | transcript variant 2, mRNA. | | |
| NM_018319 | Homo sapiens tyrosyl-DNA phosphodiesterase 1 (TDP1), | 0.5569 | 3′UTR -X--X---- |
| | transcript variant 1, mRNA. | | |
| NM_018698 | Homo sapiens nuclear transport factor 2-like export factor 2 | 0.5286 | 3′UTR -X--------- |
| | (NXT2), mRNA. | | |
| NM_000270 | Homo sapiens nucleoside phosphorylase (NP), mRNA. | 0.4619 | 3′UTR --X- |
| NM_021144 | Homo sapiens PC4 and SFRS1 interacting protein 1 (PSIP1), | 0.4619 | 3′UTR --X- |
| | transcript variant 1, mRNA. | | |
| NM_004301 | Homo sapiens actin-like 6A (ACTL6A), transcript variant 1, | 0.4618 | 3′UTR --X |
| | mRNA. | | |
| NM_178042 | Homo sapiens actin-like 6A (ACTL6A), transcript variant 3, | 0.4618 | 3′UTR --X |
| | mRNA. | | |
| NM_177989 | Homo sapiens actin-like 6A (ACTL6A), transcript variant 2, | 0.4618 | 3′UTR --X |
| | mRNA. | | |
| NM_014711 | Homo sapiens CP110 protein (CP110), mRNA. | 0.4542 | 3′UTR ---X-------- |
| NM_016245 | Homo sapiens hydroxysteroid (17-beta) dehydrogenase 11 | 0.3918 | 3′UTR ---X- |
| | (HSD17B11), mRNA. | | |
| NM_018204 | Homo sapiens cytoskeleton associated protein 2(CKAP2), | 0.3790 | 3′UTR ------X- |
| | transcript variant 1, mRNA. | | |
| NM_020963 | Homo sapiens Mov10, Moloney leukemia virus 10, homolog | 0.3790 | 3′UTR -X |
| | (mouse) (MOV10), transcript variant 1, mRNA. | | |
| NM_144652 | Homo sapiens leucine zipper-EF-hand containing | 0.3790 | 3′UTR -X |
| | transmembrane protein 2 (LETM2), mRNA. | | |
| NM_001790 | Homo sapiens cell division cycle 25 homolog C (S. pombe) | 0.3325 | 3′UTR X-- |
| | (CDC25C), transcript variant 1, mRNA. | | |
| NM_018124 | Homo sapiens ring finger and WD repeat domain 3 (RFWD3), | 0.3325 | 3′UTR -----X-------- |
| | mRNA. | | |
| NM_022809 | Homo sapiens cell division cycle 25 homolog C (S. pombe) | 0.3325 | 3′UTR X-- |
| | (CDC25C), transcript variant 2, mRNA. | | |
| NM_015148 | Homo sapiens PAS domain containing serine/threonine kinase | 0.3325 | 3′UTR -X- |
| | (PASK), mRNA. | | |
| NM_005680 | Homo sapiens TATA box binding protein (TBP)-associated | 0.3325 | 3′UTR X-- |
| | factor, RNA polymerase I, B, 63 kDa (TAF1B), mRNA. | | |
| NM_016004 | Homo sapiens intraflagellar transport 52 homolog | 0.3325 | 3′UTR -X |
| | (Chlamydomonas) (IFT52), mRNA. | | |
| NM_173084 | Homo sapiens tripartite motif-containing 59 (TRIM59), mRNA. | 0.3266 | 3′UTR ------X------ |
| NM_002417 | Homo sapiens antigen identified by monoclonal antibody Ki-67 | 0.3266 | 3′UTR ----------X--- |
| | (MKI67), transcript variant 1, mRNA. | | |
| NM_080626 | Homo sapiens BRI3 binding protein (BRI3BP), mRNA. | 0.3266 | 3′UTR -X- |
| NM_001018109 | Homo sapiens pirin (iron-binding nuclear protein) (PIR), | 0.3266 | 3′UTR X- |
| | transcript variant 2, mRNA. | | |
| NM_145663 | Homo sapiens DBF4 homolog B (S. cerevisiae) (DBF4B), | 0.3266 | 3′UTR ---X-- |
| | transcript variant 1, mRNA. | | |
| NM_052827 | Homo sapiens cyclin-dependent kinase 2 (CDK2), transcript | 0.3266 | 3′UTR ---X--- |
| | variant 2, mRNA. | | |
| NM_173529 | Homo sapiens chromosome 18 open reading frame 54 | 0.3266 | 3′UTR ---------X---------- |
| | (C18orf54), mRNA. | | |
| NM_017770 | Homo sapiens elongation of very long chain fatty acids | 0.3266 | 3′UTR --X------------- |
| | (FEN1/Elo2, SUR4/Elo3, yeast)-like 2 (ELOVL2), mRNA. | | |
| NM_001002800 | Homo sapiens structural maintenance of chromosomes 4 | 0.3266 | 3′UTR --X---- |
| | (SMC4), transcript variant 2, mRNA. | | |
| NM_012310 | Homo sapiens kinesin family member 4A (KIF4A), mRNA. | 0.3266 | 3′UTR --X- |
| NM_006636 | Homo sapiens methylenetetrahydrofolate dehydrogenase | 0.3266 | 3′UTR -X---- |
| | (NADP+ dependent) 2, methenyltetrahydrofolate cyclohydrolase | | |
| | (MTHFD2), nuclear gene encoding mitochondrial protein, | | |
| | transcript variant 1, mRNA. | | |
| NM_005496 | Homo sapiens structural maintenance of chromosomes 4 | 0.3266 | 3′UTR --X---- |
| | (SMC4), transcript variant 1, mRNA. | | |
| NM_014573 | Homo sapiens transmembrane protein 97 (TMEM97), mRNA. | 0.3266 | 3′UTR ------X---- |
| NM_014508 | Homo sapiens apolipoprotein B mRNA editing enzyme, | 0.3266 | 3′UTR -X- |
| | catalytic polypeptide-like 3C (APOBEC3C), mRNA. | | |
| NM_003662 | Homo sapiens pirin (iron-binding nuclear protein) (PIR), | 0.3266 | 3′UTR X- |
| | transcript variant 1, mRNA. | | |
| NM_001798 | Homo sapiens cyclin-dependent kinase 2 (CDK2), transcript | 0.3266 | 3′UTR ---X--- |
| | variant 1, mRNA. | | |
| NM_018101 | Homo sapiens cell division cycle associated 8 (CDCA8), | 0.3266 | 3′UTR -----X-- |
| | mRNA. | | |
| NM_024792 | Homo sapiens family with sequence similarity 57, member A | 0.2949 | 3′UTR X------ |
| | (FAM57A), mRNA. | | |
| NM_006611 | Homo sapiens killer cell lectin-like receptor subfamily A, | 0.2617 | 3′UTR ---X--- |
| | member 1 (KLRA1), mRNA. | | |
| NM_207418 | Homo sapiens gastric cancer up-regulated-2 (GCUD2), mRNA. | 0.2492 | 3′UTR --X-- |
| NM_003526 | Homo sapiens histone cluster 1, H2bc (HIST1H2BC), mRNA. | 0.2322 | 3′UTR X |
| NM_014321 | Homo sapiens origin recognition complex, subunit 6 like (yeast) | 0.2322 | 3′UTR X---- |
| | (ORC6L), mRNA. | | |
| NM_005327 | Homo sapiens hydroxyacyl-Coenzyme A dehydrogenase | 0.2303 | 3′UTR X---- |
| | (HADH), nuclear gene encoding mitochondrial protein, mRNA. | | |
| NM_001269 | Homo sapiens regulator of chromosome condensation 1 (RCC1), | 0.2303 | 3′UTR -----X |
| | transcript variant 3, mRNA. | | |
| NM_015341 | Homo sapiens non-SMC condensin I complex, subunit H | 0.2303 | 3′UTR ---------X-- |
| | (NCAPH), mRNA. | | |
| NM_133487 | Homo sapiens RAD51 homolog (RecA homolog, E. coli) (S. | 0.2303 | 3′UTR ----X- |
| | cerevisiae) (RAD51), transcript variant 2, mRNA. | | |
| NM_002875 | Homo sapiens RAD51 homolog (RecA homolog, E. coli) (S. | 0.2303 | 3′UTR ----X- |
| | cerevisiae) (RAD51), transcript variant 1, mRNA. | | |
| NM_024745 | Homo sapiens SHC SH2-domain binding protein 1 (SHCBP1), | 0.2303 | 3′UTR -----X- |
| | mRNA. | | |
| NM_003390 | Homo sapiens WEE1 homolog (S. pombe) (WEE1), mRNA. | 0.0751 | 3′UTR -----X- |

(*) Expected number of seed matches under evolutionary selective pressure

Induction of miR-1/206 Expression Blocks the Growth of RMS Xenografts in vivo by Promoting their Terminal Differentiation.

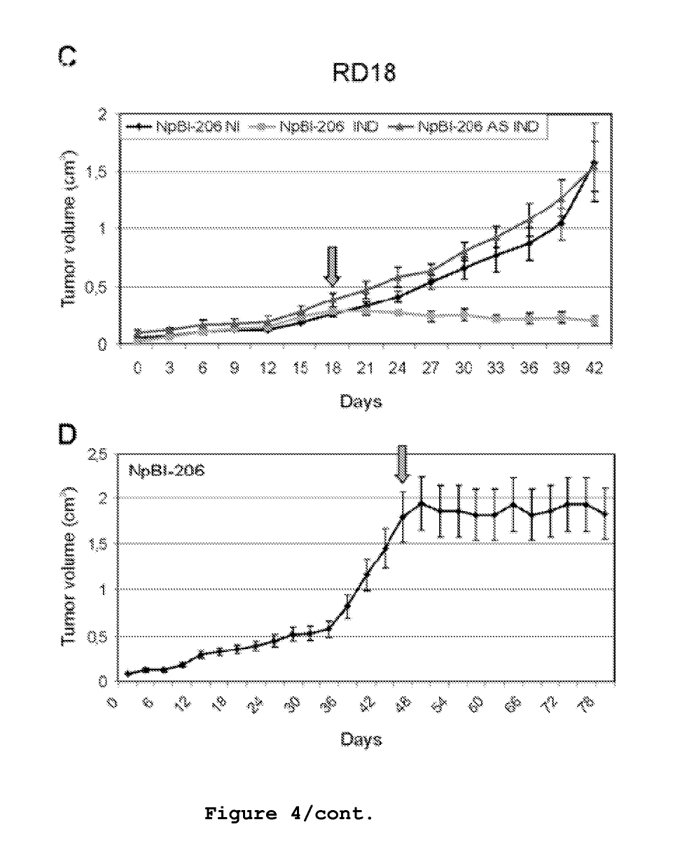

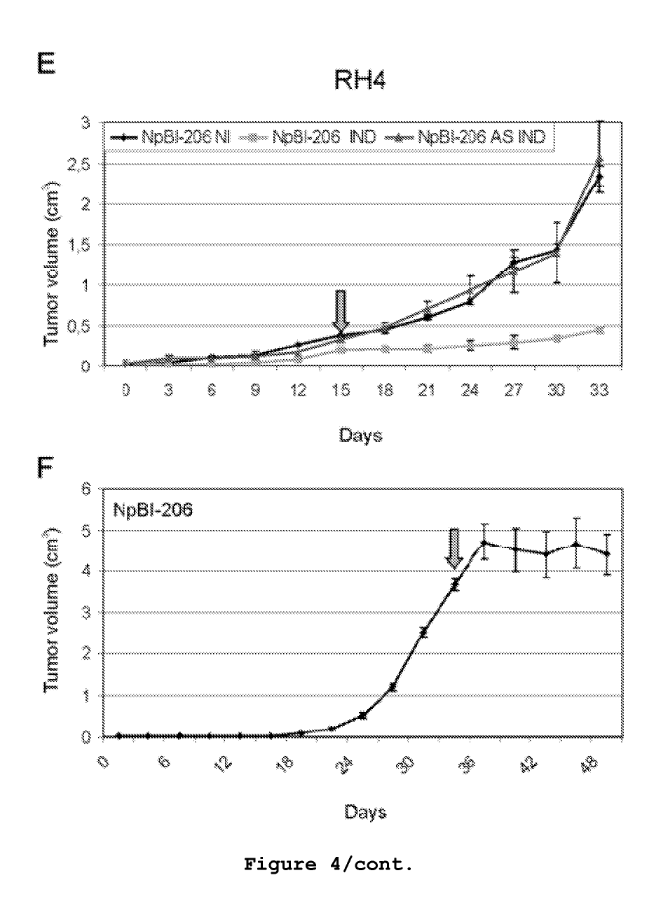

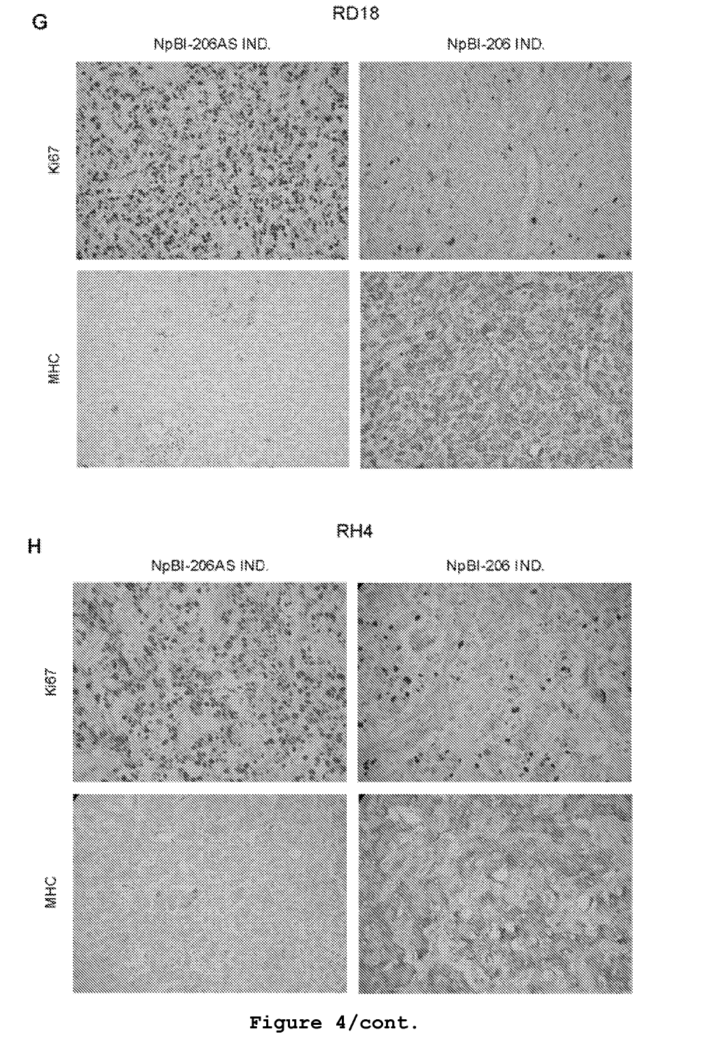

These results suggested that by tilting the balance of RMS cells toward differentiation, miR-206 could act as a tumor suppressor in vivo. To assess whether induction of miR-206 could prevent tumor growth, lentiviral-transduced ERMS and ARMS cells were injected into immuno-compromised mice kept either in inducing or non-inducing conditions (see legend of FIG. 4). Both ERMS and ARMS cells, after a slightly different lag time, formed rapidly growing tumors in animals where miR-206 was not induced. Continuous miR-206 expression essentially suppressed the growth of both types of tumor (FIGS. 4A and B). To assess whether miR-206 could have therapeutic potential for RMS treatment, its expression was induced in vivo when the tumors reached ˜0.4 cm³ in volume and after the tumors became much larger. While no effect was observed upon induction of the control antisense (AS) construct, in all four cases miR-206 expression efficiently blocked tumor growth (FIGS. 4C, D, E and F). Histological analysis (not shown), revealed a striking change in the morphology of the tumor cells, indicating a massive switch to the differentiated phenotype. The switch was confirmed by immunohistochemistry with Ki67 and MHC-specific antibodies which showed that most cells were no longer replicating (Ki67-negative) and appeared to be terminally differentiated (MHC-positive) (FIGS. 4G and H).

Met is Post-transcriptionally Downregulated by miR-1/206 During Myogenic Differentiation, and is Silenced Following Rescue of miR-1/206 Expression in RMS Cells.

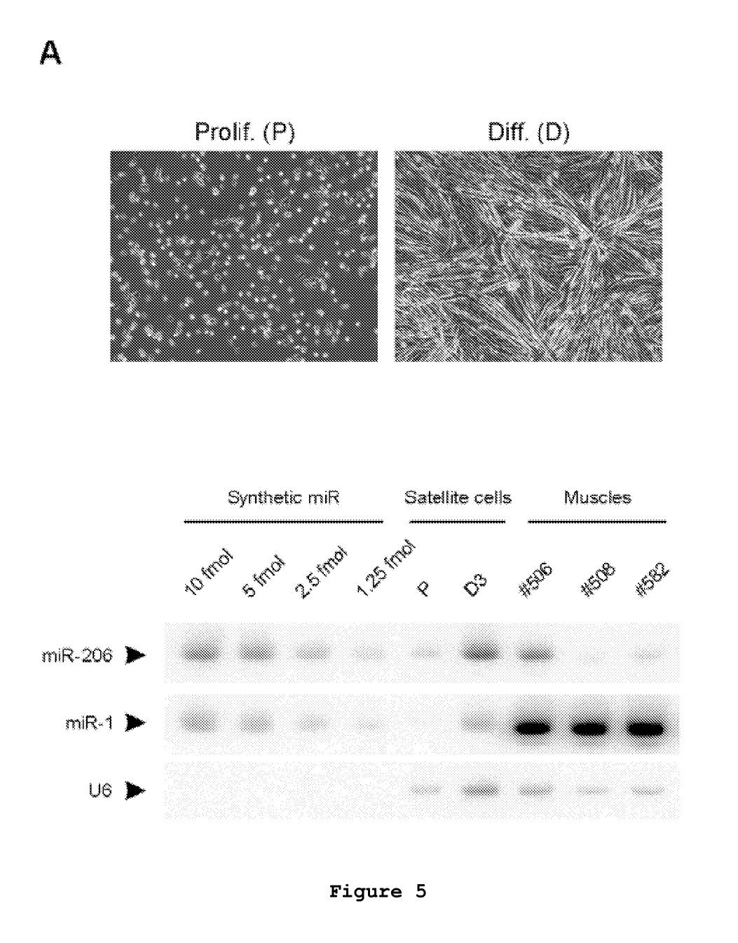

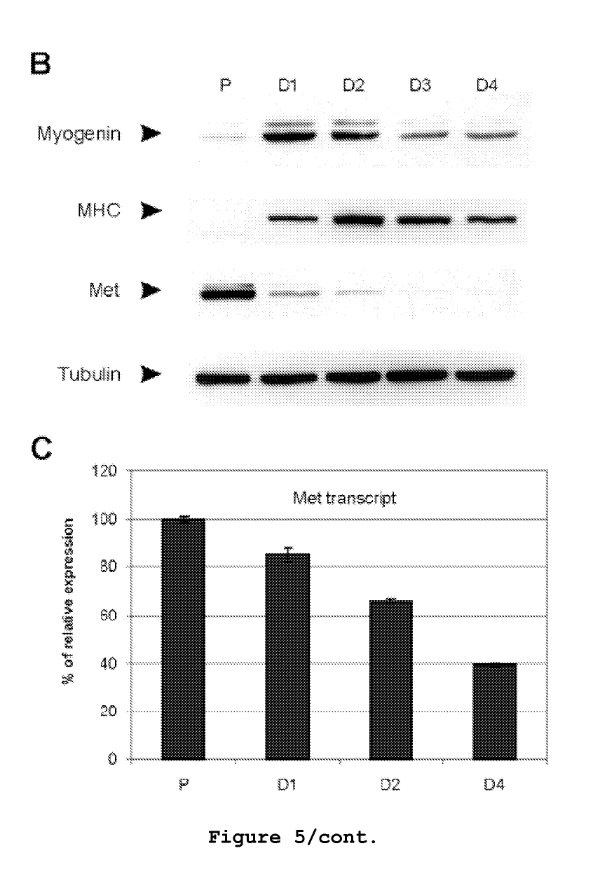

There are several potential targets of miR-1/206, which could contribute to the malignant phenotype of RMS cells. The present inventors focused on Met, a tyrosine kinase receptor overexpressed in primary RMS tumors and cell lines, which has been implicated in RMS pathogenesis. Physiologically, Met is rapidly downregulated at the onset of myogenic differentiation. To assess whether this process involves post-transcriptional mechanisms, murine satellite cells were used. When grown in high serum satellite cells actively proliferate. However, within three to four days of switching to low serum, they differentiate into myotubes (FIG. 5A, upper panel). The present inventors found that miR-1/206 expression increased concomitantly (FIG. 5A lower panel). Myogenin (a transcription factor that directly induces muscle-specific genes) and MHC (one of its targets and a marker of terminal differentiation) were rapidly upregulated (FIG. 5B). Conversely, the Met protein was almost completely downregulated from day 1 after the switch, with total depletion by day 3 (FIG. 5B). In contrast, downregulation of the Met transcript followed a much slower kinetic. In fact, at day 4 of differentiation, Met mRNA was still present at 40% of the original level (FIG. 5C). These results indicate that the Met protein is post-transcriptionally downregulated in myogenic cells at the onset of differentiation.

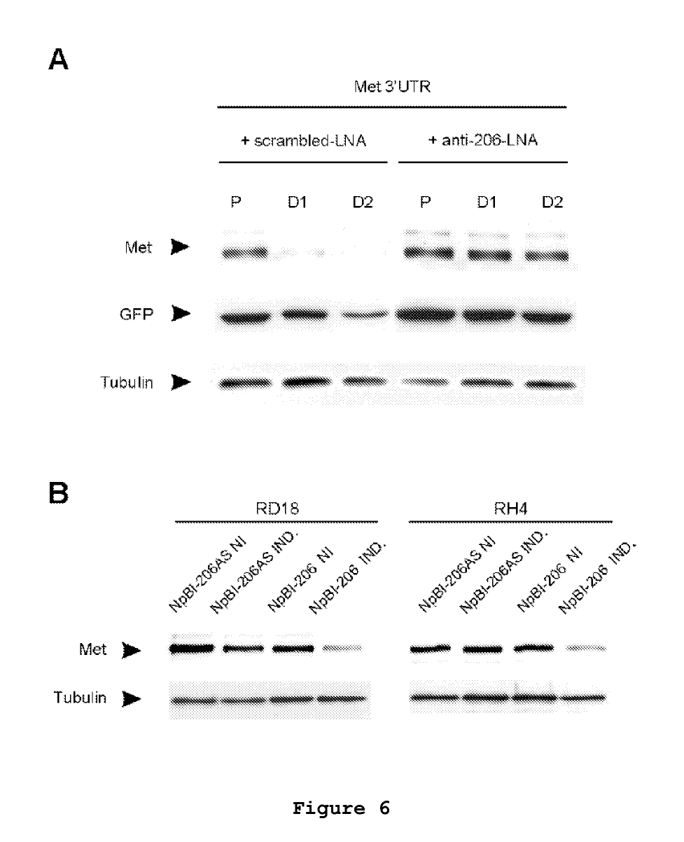

The Met transcript has two conserved binding sites for miR-1/206 in its 3′UTR. To verify whether endogenous miR-1/206 could be responsible for the rapid downregulation of Met observed upon switching to low serum, satellite cells with a reporter vector expressing GFP linked to the Met 3′UTR were transfected. Upon switch to low serum, when expression of endogenous miR-1/206 is induced (FIG. 5A), a decrease of both endogenous Met and of the transfected GFP protein (FIG. 6A) was observed. This effect was specifically abrogated by the LNA complementary to miR-1/206, indicating that in differentiating satellite cells miR-1/206 downregulates Met by binding directly to its 3′UTR. It should be noted that the LNA complementary to miR-1/206 also impaired morphological differentiation of the cells.

Finally, it has been verified whether the level of miR-1/206 expression obtained with the inducible lentiviral vector was sufficient to suppress the Met protein in ERMS and ARMS cells. In both types of RMS, induction of miR-1/206 caused a significant reduction of the Met protein (FIG. 6B), in concomitance to the enhancement of differentiation. On the other hand, expressing the constitutively active form of the receptor (Tpr-Met) in the same cells where miR-1/206 was induced, significantly lowered the number of MHC-positive cells (FIG. 9). This indicates that Met downregulation by miR-1/206 is a pre-requisite for myogenic differentiation.

1. microRNA for use in differentiation treatment of sarcoma for converting said sarcoma cells into terminally differentiated myogenic cells, wherein said microRNA is a muscle-enriched/specific microRNA, and wherein said muscle-enriched/specific microRNA is selected among microRNA-1, microRNA-206 and combinations thereof.

2. microRNA according to claim 1, wherein said sarcoma is rhabdomyosarcoma, synovial sarcoma, alveolar soft part sarcoma, liposarcoma, osteosarcoma.

3. microRNA according to claim 1, wherein said microRNA is suitable to be administered to sarcoma cells.

4. microRNA according to claim 1, wherein said microRNA is suitable to stop proliferation of sarcoma cells.

5. microRNA according to claim 1, wherein said microRNA is suitable to induce myogenic differentiation of sarcoma cells.

6. microRNA according to claim 1, wherein said microRNA is suitable to down-regulate Met gene expression.

7. microRNA according to claim 1, wherein said microRNA is suitable to be delivered by lipid nanoparticles, liposomes or a vector encoding said microRNA.

8. microRNA according to claim 7, wherein said vector is selected among lentiviral, adenoviral, or adeno-associated vectors.
